(19) United States
(12) Patent Application Publication
(10) Pub. No.: US 2023/0305009 A1
(43) Pub. Date: Sep. 28, 2023

(54) BIOMARKER COMBINATIONS FOR DETERMINING AGGRESSIVE PROSTATE CANCER

(71) Applicant: MINOMIC INTERNATIONAL LTD., Macquarie Park, New South Wales (AU)

(72) Inventors: Douglas CAMPBELL, Cremorne, New South Wales (AU); Thao Ho LE, Hawthorn, Victoria (AU); Yanling LU, Asquith, New South Wales (AU); Bradley WALSH, East Lindfield, New South Wales (AU)

(21) Appl. No.: 18/010,108

(22) PCT Filed: Jun. 30, 2021
(86) PCT No.: PCT/AU2021/050705

§ 371 Date: Dec. 13, 2022

(30) Foreign Application Priority Data
Jun. 30, 2020 (AU) 2020902212

(51) Int. Cl. G01N 33/574; G16B 25/10; G16H 50/30; G16H 50/70

(52) U.S. Cl. CPC G01N 33/57434; G16B 25/10; G16H 50/30; G16H 50/70

(57) ABSTRACT

The present invention provides methods for the diagnosis of aggressive prostate cancer, including, but not limited to, methods for discerning between aggressive and non-aggressive forms of prostate cancer, and methods for detecting aggressive prostate cancer based on comparisons to a mixed control population of subjects with non-aggressive prostate cancer or not having prostate

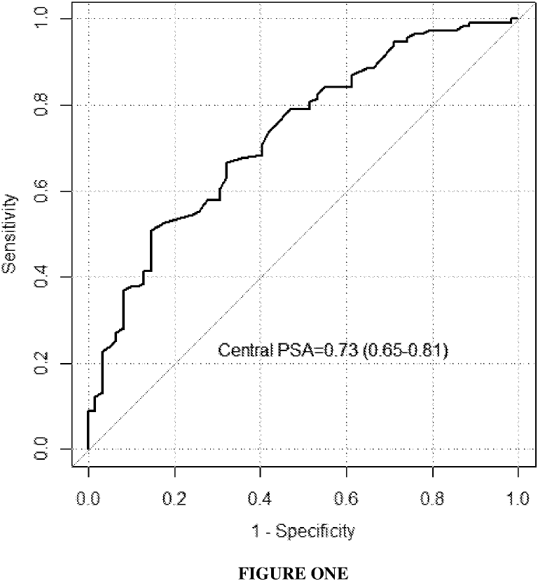

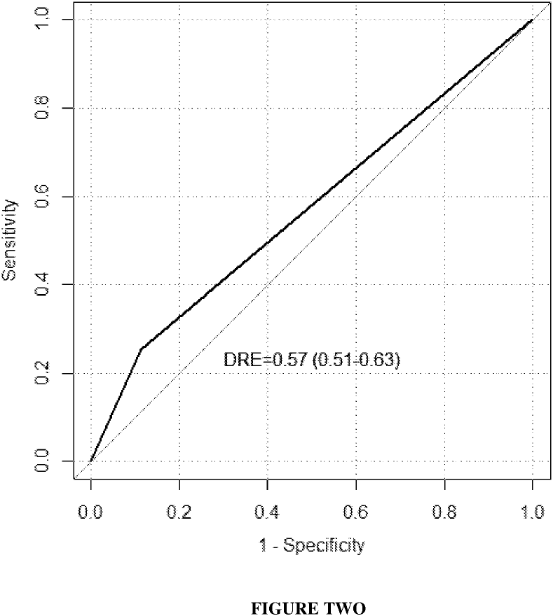

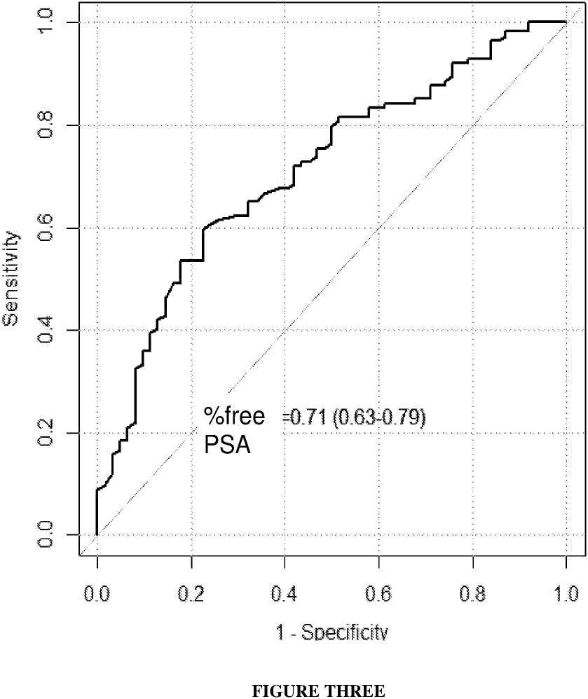

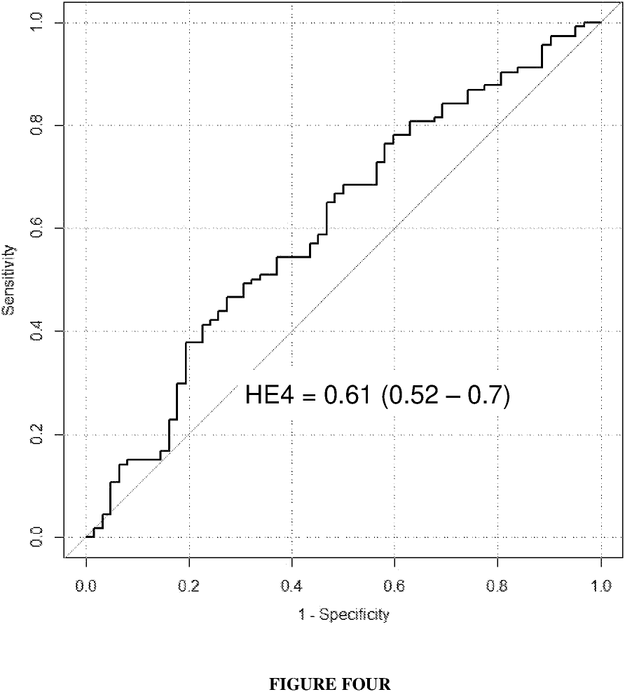

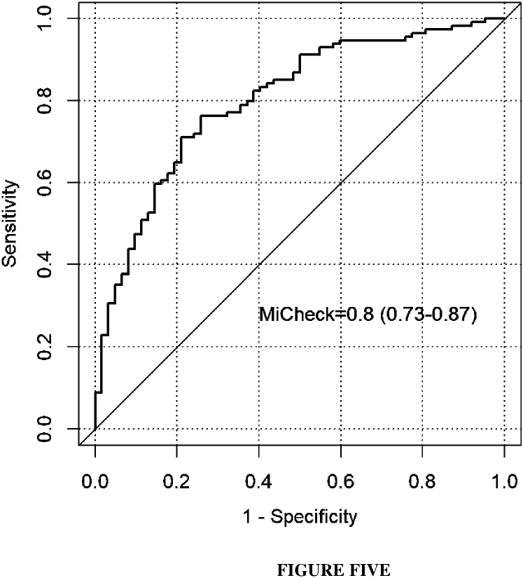

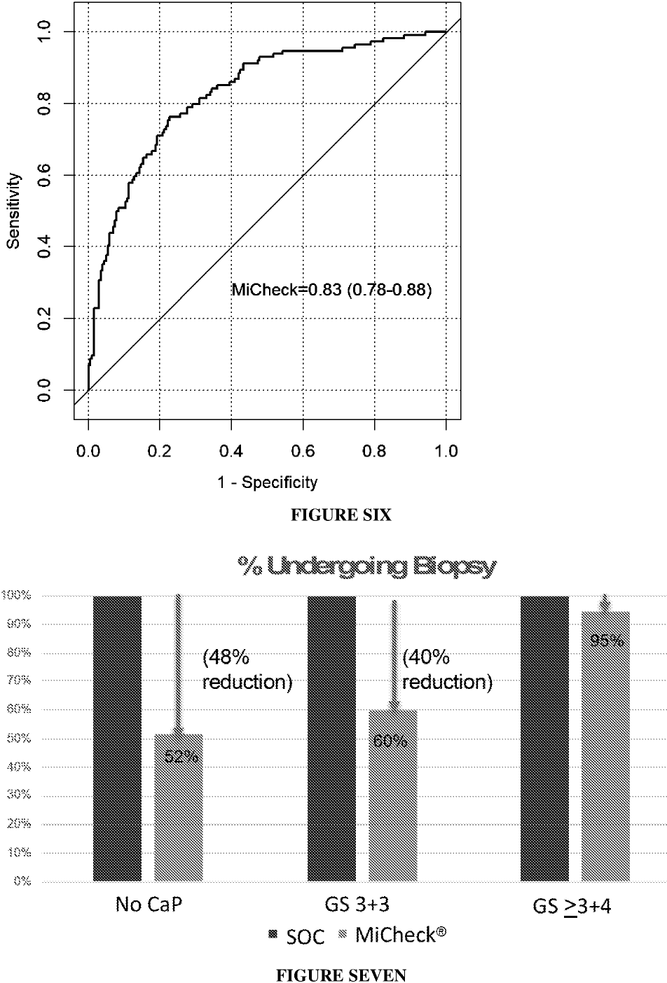

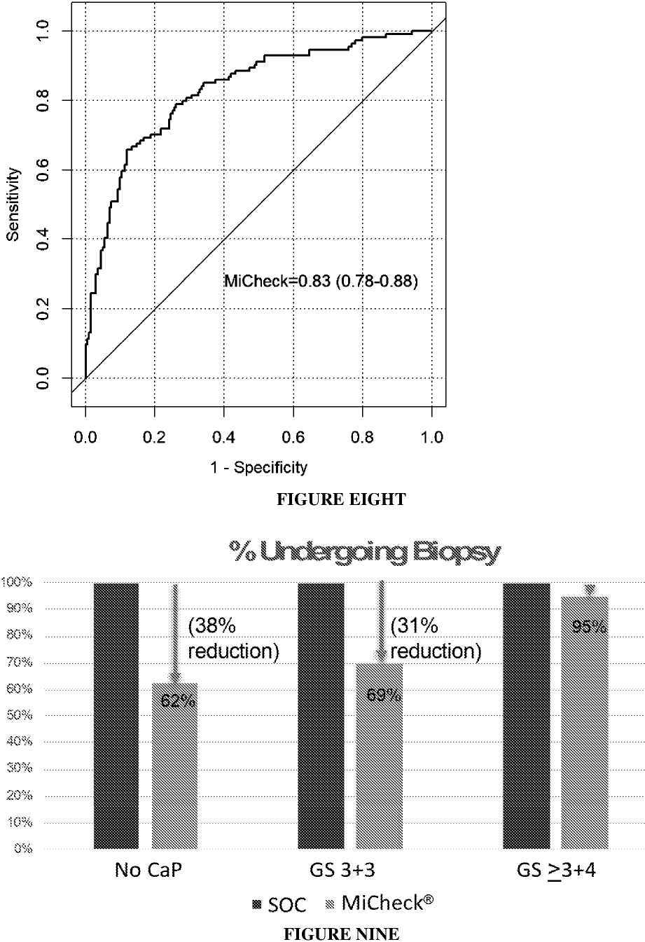

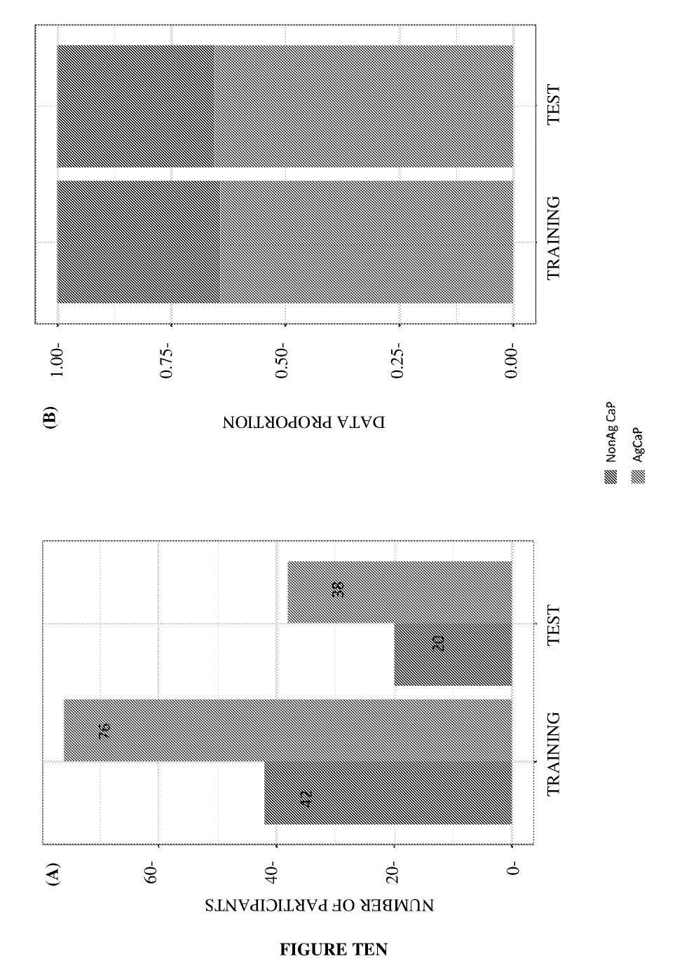

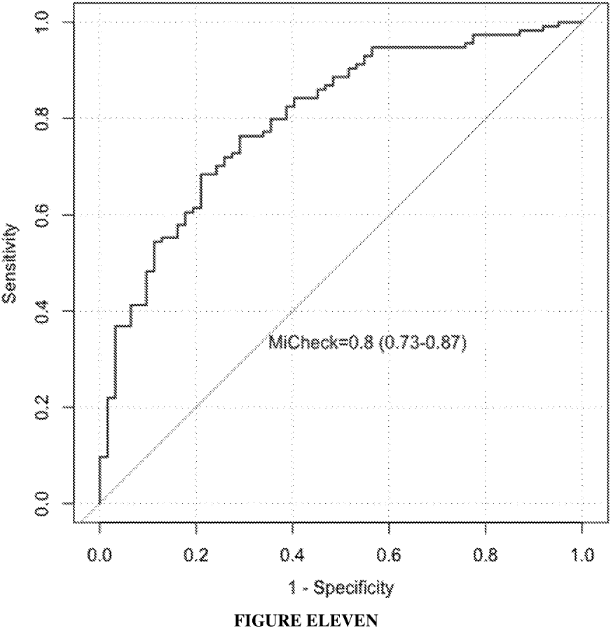

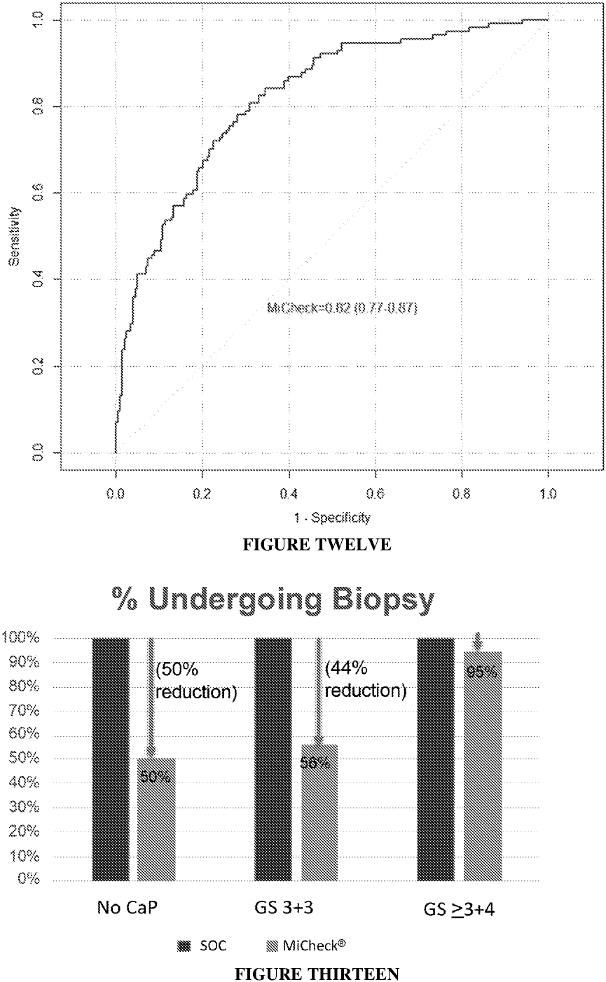

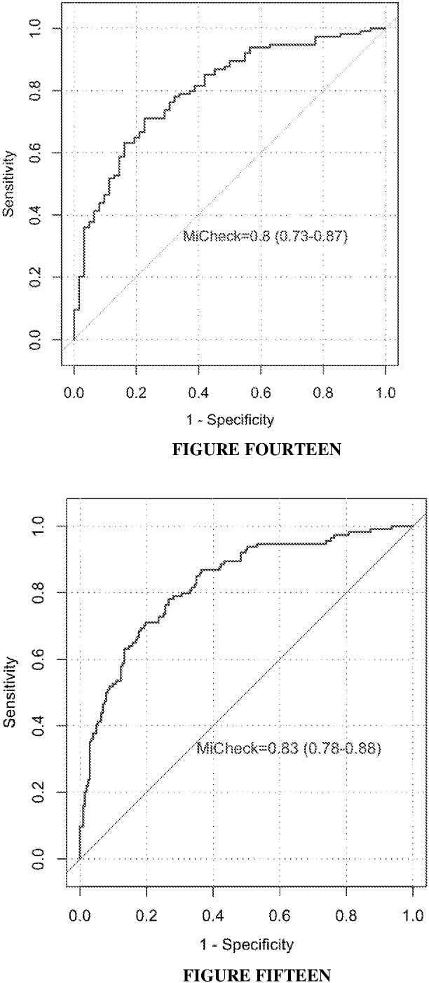

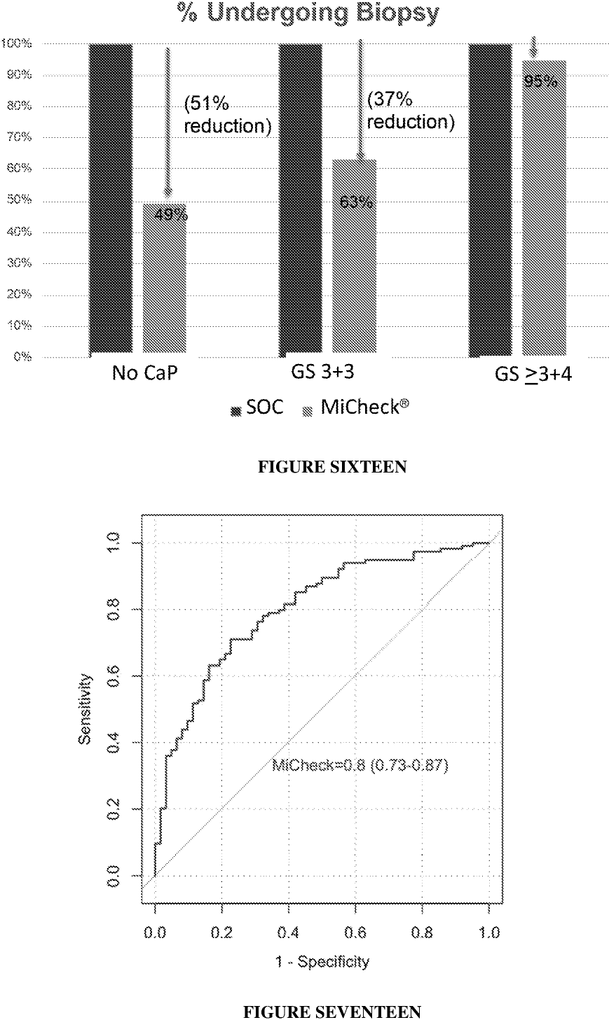

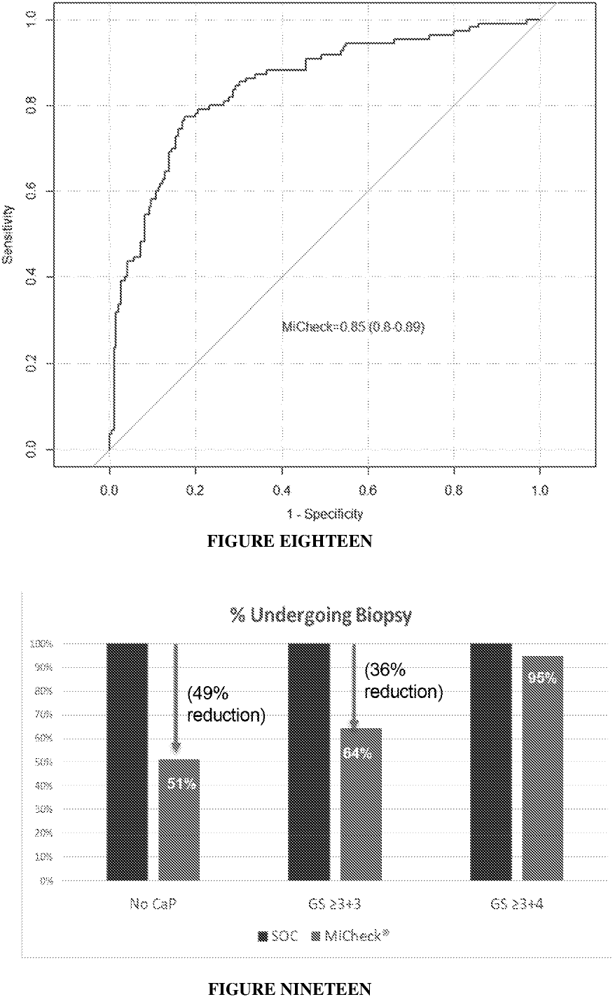

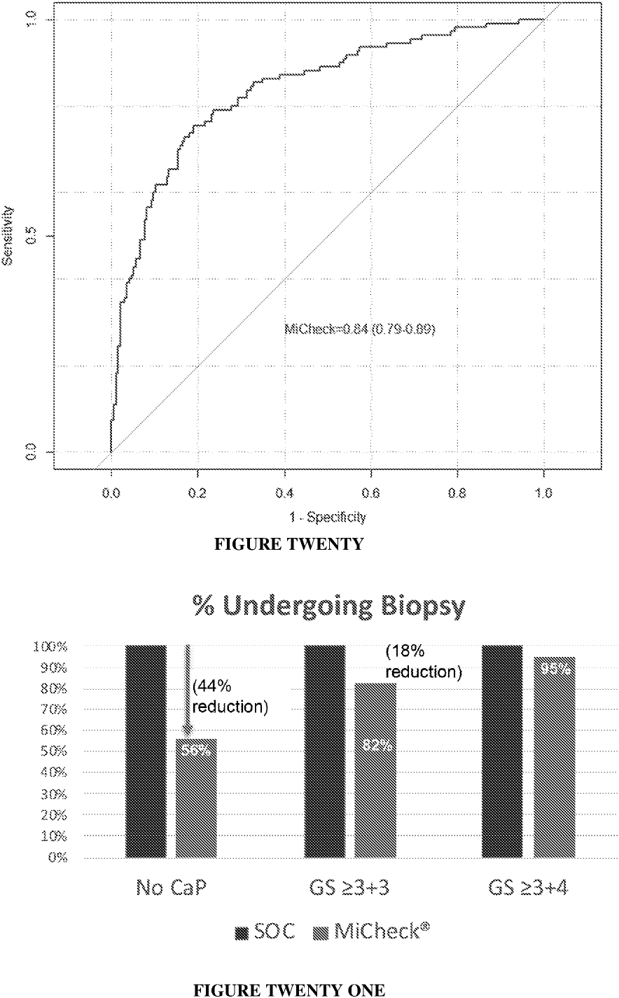

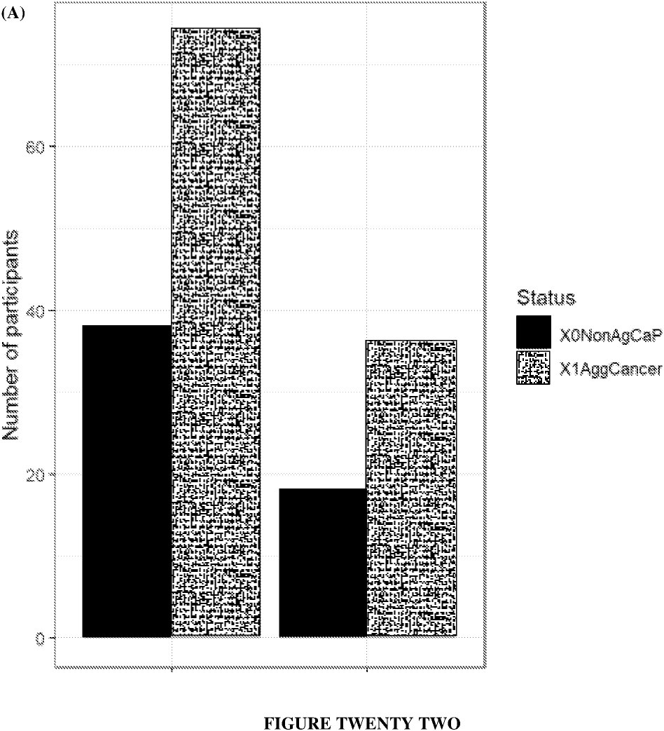

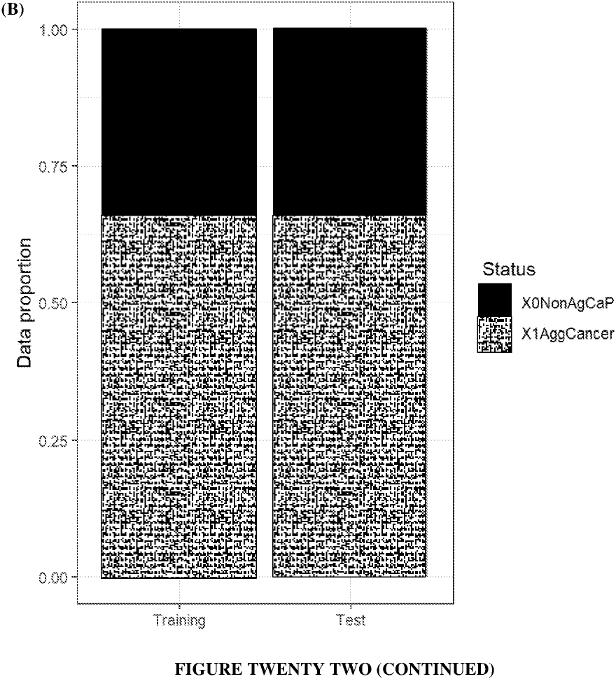

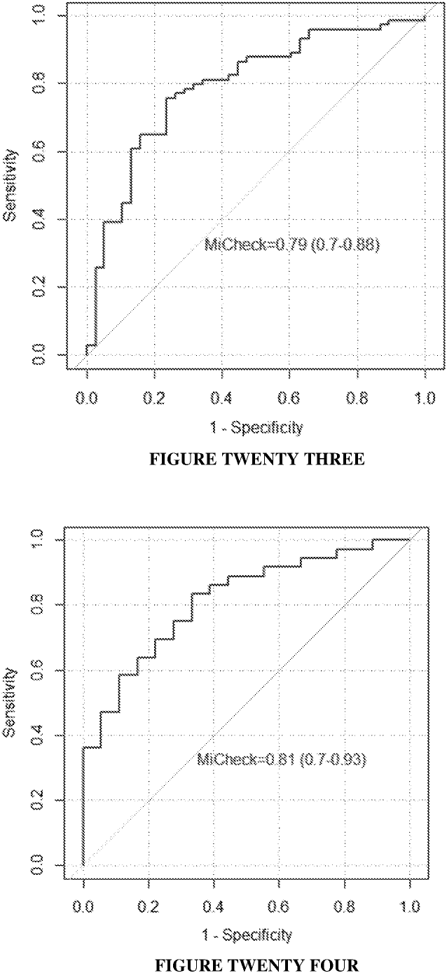

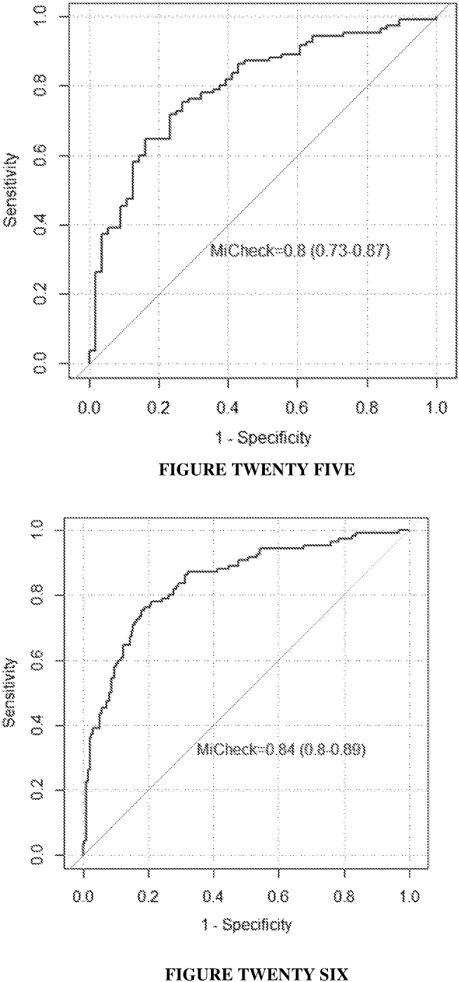

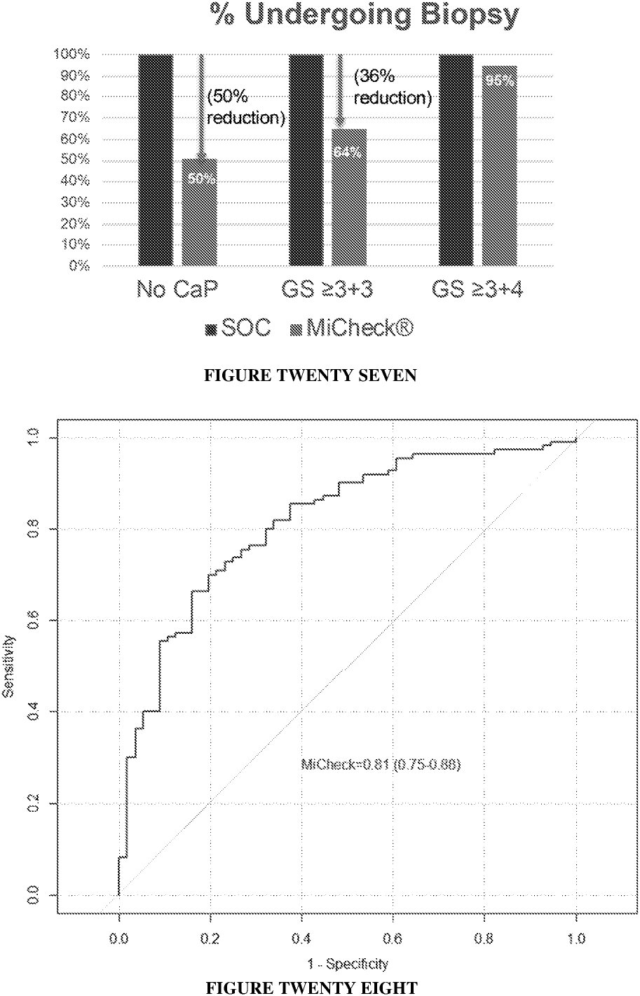

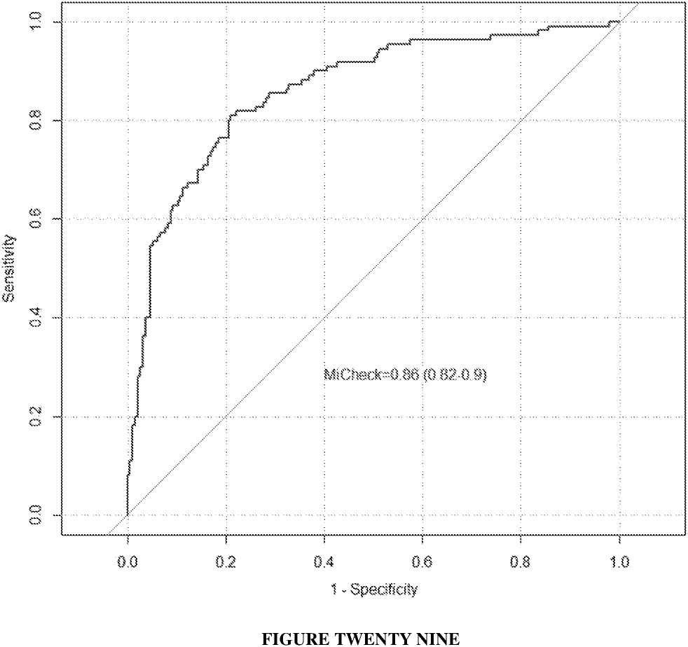

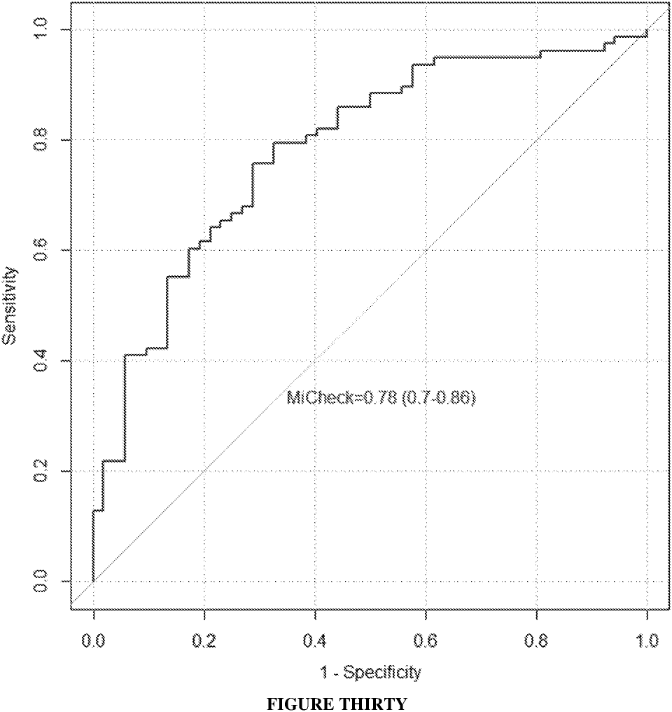

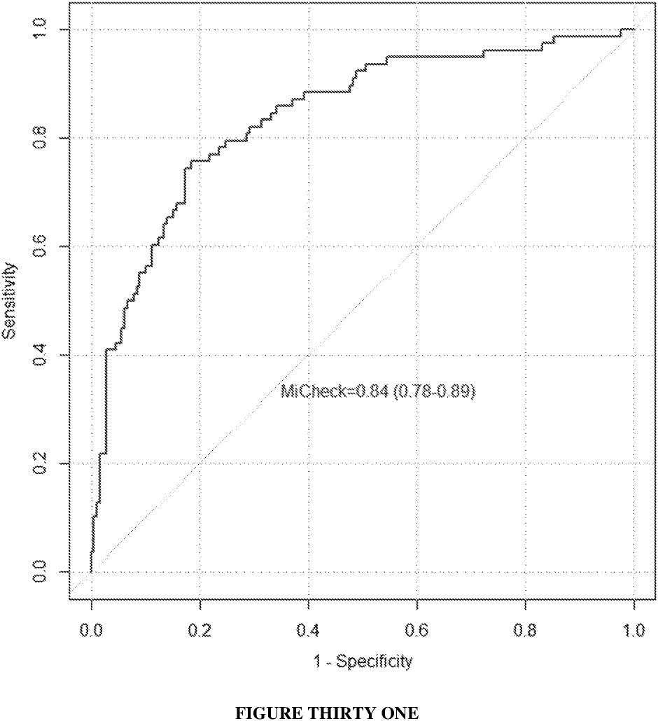

### INCORPORATION BY CROSS-REFERENCE

The present application claims priority from Australian provisional patent application number 2020902212 filed on 30 Jun. 2020, the entire content of which is incorporated herein by cross-reference.

### TECHNICAL FIELD

The present invention relates generally to the fields of immunology and medicine. More specifically, the present invention relates to the diagnosis of aggressive and non-aggressive forms of prostate cancer in subjects by assessing various combinations of biomarker/s and clinical variable/s.

### BACKGROUND

Prostate cancer is the most frequently diagnosed visceral cancer and the second leading cause of cancer death in males. According to the National Cancer Institute’s SEER program and the Centers for Disease Control’s National Center for Health Statistics, 164,690 cases of prostate cancer are estimated to have arisen in 2018 (9.5% of all new cancer cases) with an estimated 29,430 deaths (4.8% of all cancer deaths) (see SEER Cancer Statistics Factsheets: Prostate Cancer. National Cancer Institute. Bethesda, MD, http://seer.cancer.gov/statfacts/html/prost.html). The relative proportion of aggressive prostate cancers (defined as Gleason 3+4 or higher) to non-aggressive prostate cancers (defined as Gleason 3+3 or lower) differs between studies. A recent study of 1012 US men proceeding to prostate biopsy with elevated PSA demonstrated 542 men were negative for prostate cancer on biopsy, 239 had Gleason 3+3 prostate cancer and 231 had Gleason 3+4 or higher prostate cancer (Parekh et al. Eur Urol. 2015 Sep;68(3):464-70).

Commonly used screening tests for prostate cancer include digital rectal exam (DRE) and detection of prostate specific antigen (PSA) in blood. DRE is invasive and imprecise, and the prevalence of false negative (i.e. cancer undetected) and false positive (i.e. indication of cancer where none exists) results from PSA assays is well documented. Upon a positive diagnosis with DRE or PSA screening, confirmatory diagnostic tests include transrectal ultrasound, biopsy, and transrectal magnetic resonance imaging (MRI) biopsy. These techniques are invasive and cause significant discomfort to the subject under examination.

In 2012, the United States Preventative Services Taskforce (USPTF) issued a recommendation against routine prostate cancer screening using the PSA test. This led to a decrease in the number of men proceeding to biopsy following elevated PSA test results and an increase in the proportion of men presenting with aggressive prostate cancer (Fleshner & Carlsson, Nature Reviews Urology, volume 15, pages 532-534, 2018).

A general need exists for more convenient, reliable and/or accurate diagnostic tests capable of discerning between aggressive and non-aggressive forms of prostate cancer and for detecting aggressive prostate cancer.

### SUMMARY OF THE INVENTION

The present inventors have identified combinations of biomarker/s and clinical variable/s effective for detecting aggressive prostate cancer. Accordingly, the biomarker/clinical variable combinations disclosed herein can be used to detect the presence or absence of aggressive prostate cancer in a subject.

The present invention relates at least to the following series of numbered embodiments below:

Embodiment 1. A method for diagnosing aggressive prostate cancer (CaP) in a test subject, comprising:
(a) obtaining an analyte level for one or more analytes in the test subject’s biological sample, and obtaining a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject has aggressive CaP by comparison of the subject test score value and the threshold value, wherein:
   the one or more analyte/s comprise or consist of WAP four-disulfide core domain protein 2 (WFDC2 (HE4)), and optionally total prostate surface antigen (PSA),
   the one or more clinical variables comprise at least one of: %Free PSA, DRE, Prostate Volume (PV), and
   the threshold value was determined by:
   measuring said one or more analyte/s in a series of biological samples obtained from a population of subjects having aggressive CaP and from a population of control subjects not having aggressive CaP, to thereby obtain an analyte level for each said analyte in each said biological sample of the series;
   combining each said analyte level of the series with measurements of said one or more clinical variables obtained from each said subject of the populations, in a manner that allows discrimination between aggressive CaP and an absence of aggressive CaP, to thereby generate the threshold value.

Embodiment 2. The method of embodiment 1, wherein the population of control subjects comprises subjects that do not have prostate cancer and subjects that have non-aggressive prostate cancer

Embodiment 3. A method for discerning whether a test subject has non-aggressive or aggressive prostate cancer (CaP), comprising:
(a) obtaining an analyte level for one or more analytes in the test subject’s biological sample, and obtaining a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject has aggressive CaP by comparison of the subject test score value and the threshold value, wherein:
   the one or more analyte/s comprise or consist of WFDC2 (HE4), and optionally total PSA,
   the one or more clinical variables comprise at least one of: %Free PSA, DRE, Prostate Volume (PV), and
   the threshold value was determined by:
   measuring said one or more analyte/s in a series of biological samples obtained from a population of subjects having aggressive CaP and from a population of control subjects having non-aggressive CaP, to thereby obtain an analyte level for each said analyte in each said biological sample of the series;
   combining each said analyte level of the series with measurements of said one or more clinical variables obtained from each said subject of the populations, in a manner that allows discrimination between aggressive CaP and non-aggressive CaP, to thereby generate the threshold value.

Embodiment 4. The method of embodiment 1 or embodiment 3, wherein the population of control subjects has non-aggressive CaP as defined by a Gleason score of 3+3.

Embodiment 5. The method of any one of embodiments 1 to 4, wherein the threshold value is determined prior to performing the method.

Embodiment 6. The method of any one of embodiments 1 to 5, wherein the one or more clinical variables and the one or more analyte/s comprise or consist of any one of the following:
WFDC2 (HE4) and %Free PSA
WFDC2 (HE4) and DRE
WFDC2 (HE4) and PV
WFDC2 (HE4), %Free PSA, and DRE
WFDC2 (HE4), %Free PSA, and PV
WFDC2 (HE4), total PSA and %Free PSA
WFDC2 (HE4), total PSA and PV
WFDC2 (HE4), total PSA and DRE
WFDC2 (HE4), total PSA, %Free PSA, and PV, or
WFDC2 (HE4), total PSA, %Free PSA, and DRE.

Embodiment 7. The method of any one of embodiments 1 to 6, comprising selecting a subset of the combined analyte/s and/or clinical variable measurements to generate the threshold value.

Embodiment 8. The method of any one of embodiments 1 to 7, wherein said combining of each said analyte level of the series with said measurements of the one or more clinical variables comprises combining a logistic regression score of the clinical variable measurements and analyte level/s in a manner that maximizes said discrimination, in accordance with the formula:

$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times t r a n s f o r m e d \left(v a r i a b l {e}_{i}\right)\right) \\ P = \frac{e x p \left(L o g i t \left(P\right)\right)}{1 + e x p \left(L o g i t \left(P\right)\right)}\end{matrix} \qquad \text{(i)}$$

wherein:
P is probability of that the test subject has aggressive prostate cancer,
the coefficientᵢ is the natural log of the odds ratio of the variable,
the transformed variableᵢ is the natural log of the variableᵢ value; or
$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times \right) \\ t r a n s f o r m e d \left(v a r i a b l {e}_{i}\right) + c o e f f i c i e n {t}_{D R E} \times D R E \\ P = \frac{e x p \left(L o g i t \left(P\right)\right)}{1 + e x p \left(L o g i t \left(P\right)\right)}\end{matrix} \qquad \text{(ii)}$$
wherein:
P is probability that the test subject has aggressive prostate cancer,
the coefficientᵢ is the natural log of the odds ratio of the variable,
the transformed variableᵢ is the natural log of the variableᵢ value,
a DRE value of 1 indicates abnormal, while DRE value of 0 indicates normal.

Embodiment 9. The method of any one of embodiments 1 to 8, wherein said applying a suitable algorithm and/or transformation to the combination of the clinical variable measurements and analyte level/s comprises use of an exponential function, a logarithmic function, a power function and/or a root function.

Embodiment 10. The method according to any one of embodiments 1 to 9, wherein the suitable algorithm and/or transformation applied to the combination of the clinical variable measurements and analyte level/s of the test subject is in accordance with the formula:

$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times t r a n s f o r m e d \left(v a r i a b l {e}_{i}\right)\right) \\ P = \frac{e x p \left(L o g i t \left(P\right)\right)}{1 + e x p \left(L o g i t \left(P\right)\right)}\end{matrix} \qquad \text{(i)}$$

wherein:
P is probability of that the test subject has aggressive prostate cancer,
the coefficientᵢ is the natural log of the odds ratio of the variable,
the transformed variableᵢ is the natural log of the variableᵢ value; or
$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times \right) \\ t r a n s f o r m e d \left(v a r i a b l {e}_{i}\right) + c o e f f i c i e n {t}_{D R E} \times D R E \\ P = \frac{e x p \left(L o g i t \left(P\right)\right)}{1 + e x p \left(L o g i t \left(P\right)\right)}\end{matrix} \qquad \text{(ii)}$$
wherein:
P is probability of that the test subject has aggressive prostate cancer,
the coefficientᵢ is the natural log of the odds ratio of the variable,
the transformed variableᵢ is the natural log of the variableᵢ value,
a DRE value of 1 indicates abnormal, while DRE value of 0 indicates normal;
and wherein said suitable algorithm and/or transformation is used to generate the subject test score that is compared to the threshold value to thereby determine whether or not the test subject has aggressive prostate cancer.

Embodiment 11. The method according to any one of embodiments 1 to 10, wherein said combining of each said analyte level of the series with measurements of said one or more clinical variables obtained from each said subject of the populations maximizes said discrimination.

Embodiment 12. The method of any one of embodiments 1 to 11, wherein said combining of each said analyte level of the series with the measurements of one or more clinical variables obtained from each said subject of the populations is conducted in a manner that:
(i) reduces the misclassification rate between the subjects having aggressive CaP and said control subjects; and/or
(ii) increases sensitivity in discriminating between the subjects having aggressive CaP and said control subjects; and/or
(iii) increases specificity in discriminating between the subjects having aggressive CaP and said control subjects.

Embodiment 13. The method of embodiment 12, wherein said combining in a manner that reduces the misclassification rate between the subjects having aggressive CaP and said control subjects comprises selecting a suitable true positive and/or true negative rate.

Embodiment 14. The method of embodiment 12, wherein said combining in a manner that reduces the misclassification rate between the subjects having aggressive CaP and said control subjects minimizes the misclassification rate.

Embodiment 15. The method of embodiment 12, wherein said combining in a manner that reduces the misclassification rate between the subjects having aggressive CaP and said control subjects comprises minimizing the misclassification rate between the subjects having aggressive CaP and said control subjects by identifying a point where the true positive rate intersects the true negative rate.

Embodiment 16. The method of embodiment 12, wherein said selecting the threshold value from the combined clinical variable measurement/s and combined analyte level/s in a manner that increases sensitivity in discriminating between the subjects having aggressive CaP and said control subjects increases or maximizes said sensitivity.

Embodiment 17. The method of embodiment 12, wherein said selecting the threshold value from the combined clinical variable measurement/s and combined analyte level/s in a manner that increases specificity in discriminating between the subjects having aggressive CaP and said control subjects increases or maximizes said specificity.

Embodiment 18. The method according to any one of embodiments 1 to 17, wherein the one or more clinical variables and the one or more analytes comprise or consist of:
total PSA, %free PSA, DRE, WFDC2 (HE4)
total PSA, %free PSA, PV, WFDC2 (HE4), or
total PSA, %free PSA, DRE, PV, WFDC2 (HE4).

Embodiment 19. The method according to any one of embodiments 1 to 18, wherein the test subject has previously received a positive indication of prostate cancer.

Embodiment 20. The method according to any one of embodiments 1 to 19, wherein the test subject has previously received a positive indication of prostate cancer by digital rectal exam (DRE) and/or by PSA testing.

Embodiment 21. The method according to any one of embodiments 1 to 19, wherein the test subject has a PSA level of 2-10 ng/mL blood, or 4-10 ng/mL blood.

Embodiment 22. The method according to any one of embodiments 1 to 21, wherein the series of biological samples obtained from each said population and/or the test subject’s biological sample are selected from; whole blood, serum, plasma, saliva, tear/s, urine, and tissue.

Embodiment 23. The method according to any one of embodiments 1 to 22, wherein said test subject, said population of subjects having aggressive CaP, and said population of control subjects are human.

Embodiment 24. The method of any one of embodiments 1 to 23, further comprising measuring one or more analyte/s in the test subject’s biological sample to thereby obtain the analyte level for each said one or more analytes.

Embodiment 25. The method according to embodiment 24, wherein said measuring of one or more analyte/s in the test subject’s biological sample comprises:
(i) measuring one or more fluorescent signals indicative of each said analyte level;
(ii) obtaining a measurement of weight/volume of said analyte/s in the biological sample;
(iii) measuring an absorbance signal indicative of each said analyte level; or
(iv) using a technique selected from the group consisting of: electrochemiluminescence, mass spectrometry, a protein array technique, high performance liquid chromatography (HPLC), gel electrophoresis, radiolabeling, and any combination thereof.

Embodiment 26. The method according to embodiment 24 or embodiment 25, wherein the test subject’s biological sample is contacted, or the series of biological samples was contacted, with first and second antibody populations for detection of each said analyte, wherein each said antibody population has binding specificity for one of said analytes, and the first and second antibody populations have different analyte binding specificities.

Embodiment 27. The method according to embodiment 26, wherein the first and/or second antibody populations are labelled.

Embodiment 28. The method according to embodiment 27, wherein the first and/or second antibody populations comprise a label selected from the group consisting of a radiolabel, a fluorescent label, a biotin-avidin amplification system, a chemiluminescence system, microspheres, and colloidal gold.

Embodiment 29. The method according to any one of embodiments 26 to 28, wherein binding of each said antibody population to the analyte is detected by a technique selected from the group consisting of: immunofluorescence, radiolabeling, immunoblotting, Western blotting, enzyme-linked immunosorbent assay (ELISA), flow cytometry, immunoprecipitation, immunohistochemistry, biofilm test, affinity ring test, antibody array optical density test, and chemiluminescence.

Embodiment 30. The method of any one of embodiments 24 to 29, wherein said measuring of each said analyte in the biological sample from the test subject or the series of biological samples obtained from each said population comprises measuring the analytes directly.

Embodiment 31. The method of any one of embodiments 24 to 29, wherein said measuring of each said analyte in the biological sample from the test subject or the series of biological samples obtained from each said population comprises detecting a nucleic acid encoding the analytes.

Embodiment 32. The method of any one of embodiments 1 to 31, further comprising measuring the two one or more clinical variables in the test subject.

Embodiment 33. The method of any one of embodiments 1 to 32, further comprising determining said threshold value.

Embodiment 34. The method of embodiment 33, wherein determining said threshold value comprises measuring said one or more analyte/s in a series of biological samples obtained from a population of subjects having aggressive CaP and from a population of control subjects not having aggressive CaP, to thereby obtain an analyte level for each said analyte in each said biological sample of the series.

### BRIEF DESCRIPTION OF THE FIGURES

Preferred embodiments of the present invention will now be described, by way of example only, with reference to the accompanying figures wherein:

FIG. One depicts a ROC curve analysis based on PSA levels (model fitting: logistic regression) generated to differentiate [aggressive prostate cancer (AgCaP) versus non-aggressive prostate cancer (NonAgCaP)].

FIG. Two depicts depicts a ROC curve analysis based on DRE status (model fitting: logistic regression) generated to differentiate [aggressive prostate cancer (AgCaP) versus non-aggressive prostate cancer (NonAgCaP)].

FIG. Three-depicts depicts a ROC curve analysis based on %free PSA (model fitting: logistic regression) generated to differentiate [aggressive prostate cancer (AgCaP) versus non-aggressive prostate cancer (NonAgCaP)].

FIG. Four depicts a ROC curve analysis based on WFDC2 (HE4) (model fitting: logistic regression) generated to differentiate (AgCaP versus NonAgCaP).

FIG. Five depicts a ROC curve analysis based on PSA, DRE, %free PSA and WFDC2 (HE4) (model fitting: logistic regression) generated under Model 1a (AgCaP versus NonAgCaP) on the CaP population.

FIG. Six depicts a ROC curve analysis based on PSA, DRE, %free PSA and WFDC2 (HE4) (model fitting: logistic regression) generated under Model 1a (AgCaP versus NOTAgCap) on the whole evaluable population.

FIG. Seven shows a graph depicting he percentage reduction in biopsies for NoCaP, NonAgCaP and AgCaP in the whole evaluable population if a biopsy decision were made on the result of Model 1a (AgCaP versus NOT AgCap). SOC: standard of care.

FIG. Eight depicts a ROC curve analysis based on PSA, DRE, % free PSA and WFDC2 (HE4) (model fitting: logistic regression) generated under Model 1b (AgCaP versus NOT AgCap) on the whole evaluable population.

FIG. Nine shows a graph depicting the percentage reduction in biopsies for NoCaP, NonAgCaP and AgCaP in the whole evaluable population if a biopsy decision were made on the result of Model 1b (AgCaP versus NOT AgCaP). SOC: standard of care.

FIGS. Ten (A & B) shows the breakdown of NonAgCaP and AgCaP in the training and test sets used for cross-validation. Data for training set: 76 AgCaP vs 42 NonAg CaP; Data for test set: 38 AgCaP vs 20 NonAg CaP.

FIG. Eleven depicts a ROC curve analysis based on PSA, DRE, %free PSA and WFDC2 (HE4) (model fitting: cross-validated logistic regression) generated under V1 Model 1a_{vaildated} (AgCaP versus NonAgCaP) on the CaP population.

FIG. Twelve depicts a ROC curve analysis based on PSA, DRE, %free PSA and WFDC2 (HE4) (model fitting: cross-validated logistic regression) generated under V1 Model 1a_{vaildated} (AgCaP versus NOT AgCap) on the whole evaluable population.

FIG. Thirteen shows a graph depicting the percentage reduction in biopsies for NoCaP, NonAgCaP and AgCaP in the whole evaluable population if a biopsy decision were made on the result of V1 Model 1a_{vaildated} (AgCaP versus NOT AgCap). SOC: standard of care.

FIG. Fourteen depicts a ROC curve analysis based on PSA, DRE, %free PSA and WFDC2 (HE4) (model fitting: cross-validated logistic regression) generated under V2 Model 1a_{vaildated} (AgCaP versus NonAgCaP) on the CaP population.

FIG. Fifteen depicts a ROC curve analysis based on PSA, DRE, %free PSA and WFDC2 (HE4) (model fitting: cross-validated logistic regression) generated under V2 Model 1a_{vaildated} (AgCaP versus NOT AgCap) on the whole evaluable population.

FIG. Sixteen shows a graph depicting the percentage reduction in biopsies for NoCaP, NonAgCaP and AgCaP in the whole evaluable population if a biopsy decision were made on the result of V2 Model 1a_{vaildated} (AgCaP versus NOT AgCap). SOC: standard of care.

FIG. Seventeen depicts a ROC curve analysis based on PSA, PV, %free PSA and WFDC2 (HE4) (model fitting: logistic regression) generated under Model 1a (AgCaP versus NonAgCaP) on the CaP population.

FIG. Eighteen depicts a ROC curve analysis based on PSA, PV, %free PSA and WFDC2 (HE4) (model fitting: logistic regression) generated under Model 1a (AgCaP versus NonAgCaP) on the whole evaluable population.

FIG. Nineteen shows a graph depicting the percentage reduction in biopsies for NoCaP, NonAgCaP and AgCaP in the whole evaluable population if a biopsy decision were made on the result of Model 1a PSA, PV, %free PSA and WFDC2 (HE4). SOC: standard of care.

FIG. Twentydepicts a ROC curve analysis based on PSA, PV, %free PSA and WFDC2 (HE4) (model fitting: logistic regression) generated under Model 1b (AgCaP versus NonAgCaP) on the whole evaluable population.

FIG. Twenty One shows a graph depicting the percentage reduction in biopsies for NoCaP, NonAgCaP and AgCaP in the whole evaluable population if a biopsy decision were made on the result of Model 1b PSA, PV, %free PSA and WFDC2 (HE4). SOC: standard of care.

FIGS. Twenty Two (A & B) shows the breakdown of NonAgCaP and AgCaP in the training and test sets used for cross-validation of the PV model. Data for model development (training): 74 AgCaP vs 38 NonAg CaP; Data for test: 36 AgCaP vs 18 NonAg CaP. Model fitting: Logistic Regression.

FIG. Twenty Three depicts a ROC curve analysis for the training set based on PSA, PV, %free PSA and WFDC2 (HE4) for the validated model under Model 1a (AgCaP versus NonAgCaP) on the CaP population.

FIG. Twenty Four depicts a ROC curve analysis for the test set based on PSA, PV, %free PSA and WFDC2 (HE4) for the validated model under Model 1a (AgCaP versus NonAgCaP) on the CaP population.

FIG. Twenty Five depicts a ROC curve analysis based on PSA, PV, %free PSA and WFDC2 (HE4) for the validated model under Model 1a (AgCaP versus NonAgCaP) on the CaP population.

FIG. Twenty Six depicts a ROC curve analysis based on PSA, PV, %free PSA and WFDC2 (HE4) for the validated model under Model 1a (AgCaP versus NonAgCaP) on the whole evaluable population.

FIG. Twenty Seven shows a graph depicting the percentage reduction in biopsies for NoCaP, NonAgCaP and AgCaP in the whole evaluable population if a biopsy decision were made on the result of the validated PSA, PV, %free PSA and WFDC2 (HE4) model.

FIG. Twenty Eight depicts a ROC curve analysis based on PSA, PV, DRE, %free PSA and WFDC2 (HE4) (model fitting: logistic regression) generated under Model 1a (AgCaP versus NonAgCaP) on the CaP population.

FIG. Twenty Nine depicts a ROC curve analysis based on PSA, PV, DRE, %free PSA and WFDC2 (HE4) (model fitting: logistic regression) generated under Model 1a (AgCaP versus NonAgCaP) on the whole evaluable population.

FIG. Thirty depicts a ROC curve analysis based on PSA, PV, DRE, %free PSA and WFDC2 (HE4) (model fitting: logistic regression) generated under Model 1a (AgCaP versus NonAgCaP) on the CaP population with a PSA range of 2-10 ng/ml.

FIG. Thirty One depicts a ROC curve analysis based on PSA, PV, DRE, %free PSA and WFDC2 (HE4) (model fitting: logistic regression) generated under Model 1a (AgCaP versus NonAgCaP) on the whole evaluable population with a PSA range of 2-10 ng/ml.

### DEFINITIONS

As used in this application, the singular form “a”, “an” and “the” include plural references unless the context clearly dictates otherwise. For example, the phrase “an antibody” also includes multiple antibodies.

As used herein, the term “comprising” means “including.” Variations of the word “comprising”, such as “comprise” and “comprises,” have correspondingly varied meanings. Thus, for example, a biomarker/clinical variable combination “comprising” analyte A and clinical variable A may consist exclusively of analyte A and clinical variable A, or may include one or more additional components (e.g. analyte B and/or clinical variable B).

As used herein, the terms “aggressive prostate cancer” and “aggressive CaP” refer to prostate cancer with a primary Gleason score of 3 or greater and a secondary Gleason score of 4 or greater (GS>3+4).

As used herein, the terms “non-aggressive prostate cancer” and “non-aggressive CaP” refer to prostate cancer with a primary Gleason score of less than or equal to 3 and a secondary Gleason score of less than 4 (GS<3+3). Primary Gleason scores of less than 3 were not reported in the subject sample set described in this application hence the term GS3+3 is also used for non-aggressive prostate cancer.

As used herein, the terms “WFDC2” and “HE4” will be understood to refer to the same analyte (WAP Four-disulfide core domain protein 2), and can be used together or interchangeably (e.g. WFDC2 (HE4)). A non-limiting example of an WFDC2 / HE4 protein is provided under UniProtKB - Q14508 (see https://www.uniprot.org/uniprot/Q14508).

As used herein, the term “clinical variable” encompasses any factor, measurement, physical characteristic relevant in assessing prostate disease, including but not limited to: age, prostate volume, %free PSA, PSA velocity, PSA density, digital rectal examination (DRE), ethnic background, family history of prostate cancer, a prior negative biopsy for prostate cancer.

As used herein, the term “total PSA” and “Central PSA” are used interchangeably and have the same meaning, referring to a test capable of measuring free plus complexed PSA in a sample.

As used herein, the term “%free PSA” refers to the ratio of free/total PSA in a sample expressed as a percentage.

As used herein, the term “PSA level” refers to nanograms of PSA per milliliter (ng/mL) of blood in a test patient.

As used herein, the terms “biological sample” and “sample” encompass any body fluid or tissue taken from a subject including, but not limited to, a saliva sample, a tear sample, a blood sample, a serum sample, a plasma sample, a urine sample, or sub-fractions thereof.

As used herein, the terms “diagnosing” and “diagnosis” refer to methods by which a person of ordinary skill in the art can estimate and even determine whether or not a subject is suffering from a given disease or condition. A diagnosis may be made, for example, on the basis of one or more diagnostic indicators, such as for example, the detection of a combination of biomarker/s and clinical feature/s as described herein, the levels of which are indicative of the presence, severity, or absence of the condition. As such, the terms “diagnosing” and “diagnosis” thus also include identifying a risk of developing aggressive prostate cancer.

As used herein, the terms “subject” and “patient” are used interchangeably unless otherwise indicated, and encompass any animal of economic, social or research importance including bovine, equine, ovine, primate, avian and rodent species. Hence, a “subject” may be a mammal such as, for example, a human or a non-human mammal. As used herein, the term “isolated” in reference to a biological molecule (e.g. an antibody) is a biological molecule that is free from at least some of the components with which it naturally occurs.

As used herein, the terms “antibody” and “antibodies” include IgG (including IgG1, IgG2, IgG3, and IgG4), IgA (including IgA1 and IgA2), IgD, IgE, IgM, and IgY, whole antibodies, including single-chain whole antibodies, and antigen-binding fragments thereof. Antigen-binding antibody fragments include, but are not limited to, Fv, Fab, Fab′ and F(ab′)2, Fd, single-chain Fvs (scFv), single-chain antibodies, disulfide-linked Fvs (sdFv) and fragments comprising either a VL or VH domain. The antibodies may be from any animal origin or appropriate production host. Antigen-binding antibody fragments, including single-chain antibodies, may comprise the variable region/s alone or in combination with the entire or partial of the following: hinge region, CH1, CH2, and CH3 domains. Also included are any combinations of variable region/s and hinge region, CH1, CH2, and CH3 domains. Antibodies may be monoclonal, polyclonal, chimeric, multispecific, humanized, and human monoclonal and polyclonal antibodies which specifically bind the biological molecule. The antibody may be a bi-specific antibody, avibody, diabody, tribody, tetrabody, nanobody, single domain antibody, VHH domain, human antibody, fully humanized antibody, partially humanized antibody, anticalin, adnectin, or affibody.

As used herein, the terms “binding specifically” and “specifically binding” in reference to an antibody, antibody variant, antibody derivative, antigen binding fragment, and the like refers to its capacity to bind to a given target molecule preferentially over other non-target molecules. For example, if the antibody, antibody variant, antibody derivative, or antigen binding fragment (“molecule A”) is capable of “binding specifically” or “specifically binding” to a given target molecule (“molecule B”), molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level. This is commonly known as background binding and is readily discernible from molecule B-specific binding, for example, by use of an appropriate control.

As used herein, the term “kit” refers to any delivery system for delivering materials. Such delivery systems include systems that allow for the storage, transport, or delivery of reaction reagents (for example labels, reference samples, supporting material, etc. in the appropriate containers) and/or supporting materials (for example, buffers, written instructions for performing an assay etc.) from one location to another. For example, kits may include one or more enclosures, such as boxes, containing the relevant reaction reagents and/or supporting materials.

It will be understood that use of the term “between” herein when referring to a range of numerical values encompasses the numerical values at each endpoint of the range. For example, a polypeptide of between 10 residues and 20 residues in length is inclusive of a polypeptide of 10 residues in length and a polypeptide of 20 residues in length.

Any description of prior art documents herein, or statements herein derived from or based on those documents, is not an admission that the documents or derived statements are part of the common general knowledge of the relevant art. For the purposes of description all documents referred to herein are hereby incorporated by reference in their entirety unless otherwise stated.

### ABBREVIATIONS

As used herein the abbreviation “CaP” refers to prostate cancer.

As used herein the abbreviations “LG” and “HG” refer to “low grade” (i.e. Gleason 3+3) and “high grade” (i.e. Gleason 3+4 or higher) prostate cancer.

As used herein the abbreviation “PSA” refers to prostate specific antigen.

As used herein the abbreviation “WFDC2” refers to WAP Four-disulfide core domain protein 2, also known in the art as Human Epididymis Protein 4 (HE4).

As used herein the abbreviation “Acc” refers to accuracy.

As used herein the abbreviation “Sens” refers to sensitivity.

As used herein the abbreviations “Spec” or “Specs” refers to specificity.

As used herein the abbreviation “Log” refers to the natural logarithm.

As used herein the abbreviation “DRE” refers to digital rectal examination.

As used herein the abbreviation “NPV” refers to negative predictive value.

As used herein the abbreviation “PPV” refers to positive predictive value.

As used herein the abbreviation “AgCaP” refers to aggressive prostate cancer defined as prostate cancer with a Gleason score of 3+4 or greater.

As used herein the abbreviation “NonAgCaP” refers to non-aggressive prostate cancer defined as prostate cancer with a Gleason score of 3+3.

As used herein the abbreviation “NOT-AgCaP” refers to samples from subjects that do not have aggressive prostate cancer. These subjects may have non-aggressive prostate cancer or not have prostate cancer at all.

### DETAILED DESCRIPTION

The development of reliable, convenient, and accurate tests for the diagnosis of aggressive prostate cancer remains an important objective, particularly during early stages when therapeutic intervention has the highest chance of success. In particular, initial screening procedures such as DRE and PSA are unable to discern between non-aggressive and aggressive prostate cancer effectively. The present invention provides combinations of biomarker/s and clinical variables indicative of aggressive prostate cancer in subjects that may have previously been determined to have a form of aggressive prostate cancer, or alternatively be suspected of having a form of aggressive prostate cancer on the basis of one or more alternative diagnostic tests (e.g. DRE, PSA testing). The biomarker/clinical variable combinations may thus be used in various methods and assay formats to differentiate between subjects with aggressive prostate cancer and those who do not have aggressive prostate cancer including, for example, subjects with non-aggressive prostate cancer and subjects who do not have prostate cancer (e.g. subjects with benign prostatic hyperplasia and healthy subjects).

### Aggressive Prostate Cancer

The present invention provides methods for the diagnosis of aggressive prostate cancer. The methods involve detection of one or more combinations of biomarker/s and clinical variable/s as described herein.

Persons of ordinary skill in the art are well aware of standard clinical tests and parameters used to classify different prostate cancer Gleason grades and Epstein scores (see, for example, “2018 Annual Report on Prostate Diseases”, Harvard Health Publications (Harvard Medical School), 2018; the entire contents of which are incorporated herein by cross-reference).

As known to those of ordinary skill in the art, prostate cancer can be categorized into stages according to the progression of the disease. Under microscopic evaluation, prostate glands are known to spread out and lose uniform structure with increased prostate cancer progression.

By way of non-limiting example, prostate cancer progression may be categorized into stages using the AJCC TNM staging system, the Whitmore-Jewett system and/or the D′Amico risk categories. Ordinarily skilled persons in the field are familiar with such classification systems, their features and their use.

By way of further non-limiting example, a suitable system of grading prostate cancer well known to those of ordinary skill in the field is the “Gleason Grading System”. This system assigns a grade to each of the two largest areas of cancer in tissue samples obtained from a subject with prostate cancer. The grades range from 1-5, 1 being the least aggressive form and 5 the most aggressive form. Metastases are common with grade 4 or grade 5, but seldom occur, for example, in grade 3 tumors. The two grades are then added together to produce a Gleason score. A score of 2-4 is considered low grade; 5-7 intermediate grade; and 8-10 high grade. A tumor with a low Gleason score may typically grow at a slow enough rate to not pose a significant threat to the patient during their lifetime.

As known to those skilled in the art, prostate cancers may have areas with different grades in which case individual grades may be assigned to the two areas that make up most of the prostate cancer. These two grades are added to yield the Gleason score/sum, and in general the first number assigned is the grade which is most common in the tumour. For example, if the Gleason score/sum is written as ‘3+4’, it means most of the tumour is grade 3 and less is grade 4, for a Gleason score/sum of 7.

A Gleason score/sum of 3+4 and above may be indicative of aggressive prostate cancer according to the present invention. Alternatively, a Gleason score/sum of under 3+4 may be indicative of non-aggressive prostate cancer according to the present invention.

An alternative system of grading prostate cancer also known to those of ordinary skill in the field is the “Epstein Grading System”, which assigns overall grade groups ranging from 1-5. A benefit of the Epstein system is assigning a different overall score to Gleason score 7 (3+4) and Gleason score 7 (4+3) since have very different prognoses; Gleason score ‘3+4’ translates to Epstein grade group 2; Gleason score ‘4+3’ translates to Epstein grade group 3.

### Biomarker and Clinical Variable Signatures

In accordance with the methods of the present invention, aggressive prostate cancer can be discerned by a combined approach of measuring one or more clinical variables identified herein along with the levels of one or more of the biomarkers identified herein.

A biomarker as contemplated herein may be an analyte. An analyte as contemplated herein is to be given its ordinary and customary meaning to a person of ordinary skill in the art and refers without limitation to a substance or chemical constituent in a biological sample (for example, blood, cerebral spinal fluid, urine, tear/s, lymph fluid, saliva, interstitial fluid, sweat, etc.) that can be detected and quantified. Non-limiting examples include cytokines, chemokines, as well as cell-surface receptors and soluble forms thereof.

A clinical variable as contemplated herein may be associated with or otherwise indicative of prostate cancer (e.g. non-aggressive and/or aggressive forms). The clinical variable may additionally be associated with other disease/s or condition/s. Non-limiting examples of clinical variables relevant to the present invention include subject Age, prostate volume (PV), %free PSA, PSA velocity, PSA density, Prostate Health Index, digital rectal examination (DRE), ethnic background, family history of prostate cancer, prior negative biopsy for prostate cancer.

By way of non-limiting example, a combination of clinical variables and biomarkers according to the present invention can be used for discerning between non-aggressive and aggressive forms of prostate cancer, and/or for diagnosing aggressive prostate cancer based on comparisons with a mixed control population of subjects having either non-aggressive prostate cancer or no prostate cancer. The combination of clinical variables and biomarkers may comprise or consist of one, two, three, or more than three individual biomarkers, in combination with one, two, three, or more than three individual clinical variables. The biomarker/s may comprise analytes including, but not limited to WFDC2, free PSA, and/or total PSA.

Without limitation, clinical variable/s, biomarker/s and combinations thereof used for diagnosing aggressive prostate cancer in accordance with the present invention may comprise or consist of:
WFDC2 (HE4)
WFDC2 (HE4) and %Free PSA
WFDC2 (HE4) and DRE
WFDC2 (HE4), %Free PSA, and DRE
WFDC2 (HE4), total PSA and %Free PSA
WFDC2 (HE4), total PSA and DRE
WFDC2 (HE4), total PSA, %Free PSA, and DRE
total PSA, %free PSA, PV, and WFDC2 (HE4), or
total PSA, %free PSA, DRE, PV, and WFDC2 (HE4).

### Detection and Quantification of Biomarkers

A biomarker or combination of biomarkers according to the present invention may be detected in a biological sample using any suitable method known to those of ordinary skill in the art.

In some embodiments, the biomarker or combination of biomarkers is quantified to derive a specific level of the biomarker or combination of biomarkers in the sample. Level/s of the biomarker/s can be analysed according to the methods provided herein and used in combination with clinical variables to provide a diagnosis.

Detecting the biomarker/s in a given biological sample may provide an output capable of measurement, thus providing a means of quantifying the levels of the biomarker/s present. Measurement of the output signal may be used to generate a figure indicative of the net weight of the biomarker per volume of the biological sample (e.g. pg/mL; µg/mL; ng/mL etc.).

By way of non-limiting example only, detection of the biomarker/s may culminate in one or more fluorescent signals indicative of the level of the biomarker/s in the sample. These fluorescent signals may be used directly to make a diagnostic determination according to the methods of the present invention, or alternatively be converted into a different output for that same purpose (e.g. a weight per volume as set out in the paragraph directly above).

Biomarkers according to the present invention can be detected and quantified using suitable methods known in the art including, for example, proteomic techniques and techniques which utilize nucleic acids encoding the biomarkers.

Non-limiting examples of suitable proteomic techniques include mass spectrometry, protein array techniques (e.g. protein chips), gel electrophoresis, and other methods relying on antibodies having specificity for the biomarker/s including immunofluorescence, radiolabelling, immunohistochemistry, immunoprecipitation, Western blot analysis, Enzyme-linked immunosorbent assays (ELISA), fluorescent cell sorting (FACS), immunoblotting, chemiluminescence, and/or other known techniques used to detect protein with antibodies.

Non-limiting examples of suitable techniques relying on nucleic acid detection include those that detect DNA, RNA (e.g. mRNA), cDNA and the like, such as PCR-based techniques (e.g. quantitative real-time PCR; SYBR-green dye staining), UV spectrometry, hybridization assays (e.g. slot blot hybridization), and microarrays.

Antibodies having binding specificity for a biomarker according to the present invention, including monoclonal and polyclonal antibodies, are readily available and can be purchased from a variety of commercial sources (e.g. Sigma-Aldrich, Santa Cruz Biotechnology, Abcam, Abnova, R&D Systems etc.). Additionally or alternatively, antibodies having binding specificity for a biomarker according to the present invention can be produced using standard methodologies in the art. Techniques for the production of hybridoma cells capable of producing monoclonal antibodies are well known in the field. Non-limiting examples include the hybridoma method (see Kohler and Milstein, (1975) Nature, 256:495-497; Coligan et al. section 2.5.1-2.6.7 in Methods In Molecular Biology (Humana Press 1992); and Harlow and Lane Antibodies: A Laboratory Manual, page 726 (Cold Spring Harbor Pub. 1988)), the EBV-hybridoma method for producing human monoclonal antibodies (see Cole, et al. 1985, in Monoclonal Antibodies and Cancer Therapy, Alan R. Liss, Inc., pp. 77-96), the human B-cell hybridoma technique (see Kozbor et al. 1983, Immunology Today 4:72), and the trioma technique.

In some embodiments, detection/quantification of the biomarker/s in a biological sample (e.g. a body fluid or tissue sample) is achieved using an Enzyme-linked immunosorbent assay (ELISA). The ELISA may, for example, be based on colourimetry, chemiluminescence, and/or fluorometry. An ELISA suitable for use in the methods of the present invention may employ any suitable capture reagent and detectable reagent including antibodies and derivatives thereof, protein ligands and the like.

By way of non-limiting example, in a direct ELISA the biomarker of interest can be immobilized by direct adsorption onto an assay plate or by using a capture antibody attached to the plate surface. Detection of the antigen can then be performed using an enzyme-conjugated primary antibody (direct detection) or a matched set of unlabeled primary and conjugated secondary antibodies (indirect detection). The indirect detection method may utilise a labelled secondary antibody for detection having binding specificity for the primary antibody. The capture (if used) and/or primary antibodies may derive from different host species.

In some embodiments, the ELISA is a competitive ELISA, a sandwich ELISA, an in-cell ELISA, or an ELISPOT (enzyme-linked immunospot assay).

Methods for preparing and performing ELISAs are well known to those of ordinary skill in the art. Procedural considerations such as the selection and coating of ELISA plates, the use of appropriate antibodies or probes, the use of blocking buffers and wash buffers, the specifics of the detection step (e.g. radioactive or fluorescent tags, enzyme substrates and the like), are well established and routine in the field (see, for example, “The Immunoassay Handbook. Theory and applications of ligand binding, ELISA and related techniques”, Wild, D. (Ed), 4^{th} edition, 2013, Elsevier).

In other embodiments, detection/quantification of the biomarker/s in a biological sample (e.g. a body fluid or tissue sample) is achieved using Western blotting. Western blotting is well known to those of ordinary skill in the art (see for example, Harlow and Lane. Using antibodies. A Laboratory Manual. Cold Spring Harbor, New York: Cold Spring Harbor Laboratory Press, 1999; Bold and Mahoney, Analytical Biochemistry 257, 185-192, 1997). Briefly, antibodies having binding affinity to a given biomarker can be used to quantify the biomarker in a mixture of proteins that have been separated based on size by gel electrophoresis. A membrane made of, for example, nitrocellulose or polyvinylidene fluoride (PVDF) can be placed next to a gel comprising a protein mixture from a biological sample and an electrical current applied to induce the proteins to migrate from the gel to the membrane. The membrane can then be contacted with antibodies having specificity for a biomarker of interest, and visualized using secondary antibodies and/or detection reagents.

In other embodiments, detection/quantification of multiple biomarkers in a biological sample (e.g. a body fluid or tissue sample) is achieved using a multiplex protein assay (e.g. a planar assay or a bead-based assay). There are numerous multiplex protein assay formats commercially available (e.g. Bio-rad, Luminex, EMD Millipore, R&D Systems), and non-limiting examples of suitable multiplex protein assays are described in the Examples section of the present specification.

In other embodiments, detection/quantification of biomarker/s in a biological sample (e.g. a body fluid or tissue sample) is achieved by flow cytometry, which is a technique for counting, examining and sorting target entities (e.g. cells and proteins) suspended in a stream of fluid. It allows simultaneous multiparametric analysis of the physical and/or chemical characteristics of entities flowing through an optical/electronic detection apparatus (e.g. target biomarker/s quantification).

In other embodiments, detection/quantification of biomarker/s in a biological sample (e.g. a body fluid or tissue sample) is achieved by immunohistochemistry or immunocytochemistry, which are processes of localizing proteins in a tissue section or cell, by use of antibodies or protein binding agent having binding specificity for antigens in tissue or cells. Visualization may be enabled by tagging the antibody/agent with labels that produce colour (e.g. horseradish peroxidase and alkaline phosphatase) or fluorescence (e.g. fluorescein isothiocyanate (FITC) or phycoerythrin (PE)).

Persons of ordinary skill in the art will recognize that the particular method used to detect biomarker/s according to the present invention or nucleic acids encoding them is a matter of routine choice that does not require inventive input.

### Measurement of Clinical Variables

A clinical variable or a combination of clinical variables according to the present invention may be assessed/measured/quantified using any suitable method known to those of ordinary skill in the art.

In some embodiments, the clinical variable/s may comprise relatively straightforward parameter/s (e.g. age) accessible, for example, via medical records.

In other embodiments, the clinical variable/s may require assessment by medical and/or other methodologies known to those of ordinary skill in the art. For example, prostate volume may require measurement by techniques using ultrasound (e.g. transabdominal ultrasonography, transrectal ultrasonography), magnetic resonance imaging, and the like. DRE results are typically scored as normal or abnormal/suspicious.

Clinical variable/s relevant to the diagnostic methods of the present invention may be assessed, measured, and/or quantified using additional or alternative methods including, by way of example, digital rectal exam, biopsy and/or MRI fusion.

Clinical variable/s such as PSA level, free PSA, total PSA, %free PSA may be determined by use of clinical immunoassays such as the Beckman Coulter Access 2 analyzer and associated Hybritech assays, Roche Cobas, Abbott Architect or other similar assays.

### Analysis of Biomarkers, Clinical Variables and Diagnosis

According to methods of the present invention, the assessment of a given combination of clinical variable/s and biomarker/s may be used as a basis to diagnose aggressive prostate cancer, or determine an absence of aggressive prostate cancer in a subject of interest.

In relation to assessing biomarker component/s of the combination, the methods generally involve analyzing the targeted biomarker/s in a given biological sample or a series of biological samples to derive a quantitative measure of the biomarker/s in the sample. Suitable biomarker/s include, but are not limited to, those biomarkers and biomarker combinations referred to above in the section entitled “Biomarker and clinical variable signatures”, and the Examples of the present application. By way of non-limiting example only, the quantitative measure may be in the form of a fluorescent signal or an absorbance signal as generated by an assay designed to detect and quantify the biomarker/s. Additionally or alternatively, the quantitative measure may be provided in the form of weight/volume measurements of the biomarker/s in the sample/s.

Similarly, in relation to assessing clinical variable component/s of the combination, assessment of feature/s such as, for example, subject age and/or prostate volume can be made and a representative value generated (e.g. a numerical value). Suitable clinical variable/s include, but are not limited to, those clinical variable/s referred to above in the section entitled “Biomarker and clinical variable signatures”, and the Examples of the present application.

In some embodiments, the methods of the present invention may comprise a comparison of levels of the biomarker/s and clinical variable/s in patient populations known to suffer from aggressive prostate cancer, known to suffer from non-aggressive cancer, or known not to suffer from prostate cancer (e.g. benign prostatic hyperplasia patient populations and/or healthy patient populations). For example, levels of biomarker/s and measures of clinical variable/s can be ascertained from a series of biological samples obtained from patients having an aggressive prostate cancer compared to patients having a non-aggressive prostate cancer. Aggressive prostate cancer may be characterized by a minimum Gleason grade or score/sum (e.g. at least 7 (e.g. 3 + 4 or 4 + 3, 5+2), or at least 8 (e.g. 4+4, 5 + 3 or 3 + 5).

The level of biomarker/s observed in samples from each individual population and clinical variable/s of the individuals within each population may be collectively analysed to determine a threshold value that can be used as a basis to provide a diagnosis of aggressive prostate cancer, or an absence of aggressive prostate cancer. For example, a biological sample from a patient confirmed or suspected to be suffering from aggressive prostate cancer can be analysed and the levels of target biomarker/s according to the present invention determined in combination with an assessment of clinical variable/s. Comparison of levels of the biomarker/s and the clinical variable/s in the patient’s sample to the threshold value/s generated from the patient populations can serve as a basis to diagnose aggressive prostate cancer or an absence of aggressive prostate cancer.

Accordingly, in some embodiments the methods of the present invention comprise diagnosing whether a given patient suffers from aggressive prostate cancer. The patient may have been previously confirmed to have or suspected of having prostate cancer, for example, as a result of a DRE and/or PSA test. In such situations, it is advantageous for the patient to determine whether the patient is likely to have aggressive prostate cancer or not, in accordance with the methods described herein avoiding the need for a prostate biopsy.

Without any particular limitation, a diagnostic method according to the present invention may involve discerning whether a subject has or does not have aggressive prostate cancer. The method may comprise obtaining a first series of biological samples from a first group of patients biopsy-confirmed to be suffering from non-aggressive prostate cancer, and a second series of biological samples from a second group of patients biopsy-confirmed to be suffering from aggressive prostate cancer. A threshold value for discerning between the first and second patient groups may be generated by measuring clinical variable/s such as subject age and/or prostate volume and/or DRE status and detecting levels/concentrations of one, two, three, four, five or more than five biomarkers in the first and second series of biological samples to thereby obtain a biomarker level for each biomarker in each biological sample of each series. Clinical variables and prostate volume are considered “variables” in determining the presence or absence of aggressive prostate cancer. The variables may be combined in a manner that allows discrimination between samples from the first and second group of patients. A threshold value or probability score may be selected from the combined variable values in a suitable manner such as any one or more of a method that: reduces the misclassification rate between the first and second group of patients; increases or maximizes the sensitivity in discriminating between the first and second group of patients; and/or increases or maximizes the specificity in discriminating between the first and second group of patients; and/or increases or maximises the accuracy in discriminating between the first and second group of patients. A suitable algorithm and/or transformation of individual or combined variable values obtained from the test subject and its biological sample may be used to generate the variable values for comparison to the threshold value. In some embodiments, one or more variables used in deriving the threshold value and/or the test subject score are weighted.

In some embodiments, the subject may receive a negative diagnosis for aggressive prostate cancer if the subject’s score generated from the combined biomarker level/s and clinical variable/s is less than the threshold value. In some embodiments, the subject receives a positive diagnosis for aggressive prostate cancer if the subject’s score generated from the combined biomarker level/s and clinical variable/s is less than the threshold value. In some embodiments, the subject receives a negative diagnosis for aggressive prostate cancer if the subject’s score generated from the combined biomarker level/s and clinical variable/s is more than the threshold value. In some embodiments, the patient receives a positive diagnosis for aggressive prostate cancer if the subject’s score generated from the combined biomarker level/s and clinical variable/s is more than the threshold value.

Suitable and non-limiting methods for conducting these analyses are described in the Examples of the present application.

One non-limiting example of such a method is Receiver Operating Characteristic (ROC) curve analysis. Generally, the ROC analysis may involve comparing a classification for each patient tested to a ‘true’ classification based on an appropriate reference standard. Classification of multiple patients in this manner may allow derivation of measures of sensitivity and specificity. Sensitivity will generally be the proportion of correctly classified patients among all of those that are truly positive, and specificity the proportion of correctly classified cases among all of those that are truly negative. In general, a trade-off may exist between sensitivity and specificity depending on the threshold value selected for determining a positive classification. A low threshold may generally have a high sensitivity but relatively low specificity. In contrast, a high threshold may generally have a low sensitivity but a relatively high specificity. A ROC curve may be generated by inverting a plot of sensitivity versus specificity horizontally. The resulting inverted horizontal axis is the false positive fraction, which is equal to the specificity subtracted from 1. The area under the ROC curve (AUC) may be interpreted as the average sensitivity over the entire range of possible specificities, or the average specificity over the entire range of possible sensitivities. The AUC represents an overall accuracy measure and also represents an accuracy measure covering all possible interpretation thresholds.

While methods employing an analysis of the entire ROC curve are encompassed, it is also intended that the methods may be extended to statistical analysis of a partial area (partial AUC analysis). The choice of the appropriate range along the horizontal or vertical axis in a partial AUC analysis may depend at least in part on the clinical purpose. In a clinical setting in which it is important to detect the presence of aggressive prostate cancer with high accuracy, a range of relatively high false positive fractions corresponding to high sensitivity (low false negatives) may be used. Alternatively, in a clinical setting in which it is important to exclude the presence of aggressive prostate cancer, a range of relatively low false positive fractions equivalent to high specificities (high true positives) may be used.

### Subjects, Samples and Controls

A subject or patient referred to herein encompasses any animal of economic, social or research importance including bovine, equine, ovine, canine, primate, avian and rodent species. A subject or patient may be a mammal such as, for example, a human or a non-human mammal. Subjects and patients as described herein may or may not suffer from aggressive prostate cancer, or may or may not suffer from a non-aggressive prostate cancer.

In accordance with methods of the present invention, clinical variable/s of a given subject may be assessed and the output combined with levels of biomarker/s measured in a sample from the subj ect.

A sample used in accordance the methods of the present invention may be in a form suitable to allow analysis by the skilled artisan. Suitable samples include various body fluids such as blood, plasma, serum, semen, urine, tear/s, cerebral spinal fluid, lymph fluid, saliva, interstitial fluid, sweat, etc. The urine may be obtained following massaging of the prostate gland.

The sample may be a tissue sample, such as a biopsy of the tissue, or a superficial sample scraped from the tissue. The tissue may be from the prostate gland. In another embodiment the sample may be prepared by forming a suspension of cells made from the tissue.

The methods of the present invention may, in some embodiments, involve the use of control samples.

A control sample is any corresponding sample (e.g. tissue sample, blood, plasma, serum, semen, tear/s, or urine) that is taken from an individual without aggressive prostate cancer. In certain embodiments, the control sample may comprise or consist of nucleic acid material encoding a biomarker according to the present invention.

In some embodiments, the control sample can include a standard sample. The standard sample can provide reference amounts of biomarker at levels considered to be control levels. For example, a standard sample can be prepared to mimic the amounts or levels of a biomarker described herein in one or more samples (e.g. an average of amounts or levels from multiple samples) from one or more subjects, who may or may not have aggressive prostate cancer.

In some embodiments control data may be utilized. Control data, when used as a reference, can comprise compilations of data, such as may be contained in a table, chart, graph (e.g. database or standard curve) that provide amounts or levels of biomarker/s and/or clinical variable feature/s considered to be control levels. Such data can be compiled, for example, by obtaining amounts or levels of the biomarker in one or more samples (e.g. an average of amounts or levels from multiple samples) from one or more subjects, who may or may not have aggressive prostate cancer. Clinical variable control data can be obtained by assessing the variable in one or more subjects who may or may not have aggressive prostate cancer.

### Kits

Also contemplated herein are kits for performing the methods of the present invention.

The kits may comprise reagents suitable for detecting one or more biomarker/s described herein, including, but not limited to, those biomarker and biomarker combinations referred to in the section above entitled “Biomarker and clinical variable signatures”.

By way of non-limiting example, the kits may comprise one or a series of antibodies capable of binding specifically to one or a series of biomarkers described herein.

Additionally or alternatively, the kits may comprise reagents and/or components for determining clinical variable/s of a subject (e.g. PSA levels), and/or for preparing and/or conducting assays capable of quantifying one or more biomarker/s described herein (e.g. reagents for performing an ELISA, multiplex bead-based Luminex assay, flow cytometry, Western blot, immunohistochemistry, gel electrophoresis (as suitable for protein and/or nucleic acid separation) and/or quantitative PCR. Such assays may be performed using systems such as the Roche Cobas, Abbott Architect or Alinity, or Beckmann Coulter Access 2 analyzer and associated Hybritech assays.

Additionally or alternatively, the kits may comprise equipment for obtaining and/or processing a biological sample as described herein, from a subject.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific example(s), which should not be construed as in any way limiting.

### Example 1: Background & Study Design

### 1.1 Clinical Diagnostic Pathways

A flow diagram depicting a typical clinical diagnostic pathway for aggressive prostate cancer is shown in the schematic below.

[image]

In brief:

1. Primary care physician refers patient with raised PSA result to a urologist.

2. Urologist repeats PSA test.

3. If above the age-adjusted PSA cut-off, the patient proceeds to biopsy.

4. If the biopsy shows a Gleason score 3+4 (or above) treatment with various modalities such as surgery, radiation, drugs in initiated.

5. If biopsy shows Gleason score of 3+3 physician may consider transperineal biopsy, MRI or active surveillance.

The flow diagram below outlines an exemplary strategy for implementation of the diagnostic methods of the present invention.

[image]

Briefly:

1. The primary care physician refers patient with raised PSA result to a urologist.

2. The urologist repeats PSA and performs diagnostic method according to the present invention

3. If the method provides a ‘no aggressive cancer’ determination the patient does not proceed to biopsy but is followed up in 3-6 months, with possible biopsy at 1 year

5. If the method provides an aggressive diagnosis the urologist orders a biopsy. If the biopsy shows Gleason score 3+4 (or above) treat with various modalities such as surgery, radiation, drugs.

6. If the biopsy shows Gleason score of 3+3 a transperineal biopsy, MRI or active surveillance can be considered.

### 1.2 Overview of Model Development

A summary of the strategy used to identify model components follows below:
Samples were collected from a representative contemporary US patient population (‘CUSP’ prospective trial).
Samples were measured using current prostate cancer diagnosis tests: PSA, %free PSA
Measurements of clinical variables used in risk calculators were made (age, ethnic background, PSA, DRE, prostate volume, family history, prior biopsy results).
The performance of clinical tests/factors at differentiating aggressive vs non-aggressive CaP and aggressive vs NOT-aggressive CaP in this cohort were determined.
Samples were measured using a panel of multiple biomarkers.
Univariate analysis of clinical variables and individual biomarkers at differentiating aggressive vs non-aggressive CaP and aggressive vs non-aggressive CaP in this cohort was carried out.
Models were developed using existing clinical tests/factors and adding one biomarker marker (note this approach minimizes the number of new markers that need to be added to existing tests).

### 1.3 Patient Cohort and Trial Parameters

A prospective clinical trial was designed to collect a representative contemporary patient population from the United States of America. This meant that the study had representative frequencies of different ethnic groups in the USA and also reflected the contemporary prevalence of either no cancer, non-aggressive prostate cancer or aggressive prostate cancer. All patients who were recruited to the trial presented on the basis of an elevated age adjusted PSA and underwent biopsy at their local clinical site. Serum and plasma samples were collected together with a blood sample for standardized PSA test (performed in a central lab on an Abbott Architect machine). In addition to the biopsy assessment at the local site, a central biopsy review was performed by a single pathologist. The central PSA value and central biopsy classification were used for model development. The full details of the trial are described in Shore et al, Urologic Oncology April 2020 doi: 10.1016/j.urolonc.2020.03.011¹.

The prospective non-randomized case-control study was designed having primary and secondary endpoints:
Primary endpoint: detection of prostate cancer vs non-prostate cancer patients
Secondary endpoint: differentiation of aggressive (defined as Gleason ≥3+4) vs non-aggressive (defined as Gleason 3+3) prostate cancer

The study was conducted in 12 US research centers and accrued a total of 384 subjects:
Arm 1 (Healthy Normal): 52 patients
Arm 2 (Prostate Biopsy): 332 (100%) patients
   Cohort A: 148 patients (45%), no cancer
   Cohort B: 64 patients (19%), GS = 6, CaP
   Cohort C: 120 patients (36%), GS ≥ 7 (≥ 3+4), CaP

Serum and plasma samples were collected, and standardized PSA test and centralized pathology were reviewed (both Gleason Score and Epstein scores).

Inclusion criteria were as follows:
ARM 1: Healthy Normal (HN)
   Subjects 50 years or older
   Low PSA (performed at most 12 months prior) with low PSA defined as: < 1.5 ng/mL between ages 50 and 60, < 3 ng/mL above age 60
   Signed informed consent
ARM 2: Prostate Biopsy
   Subjects 40 years or older
   All subjects who were referred for or had undergone either a de novo or a repeat prostate biopsy for high PSA where high PSA was defined as: ≥ 1 ng/ml between ages 40 and 49, ≥ 2 ng/mL between ages 50 and 60, ≥ 3 ng/mL for age 60 and above
   Signed informed consent.
Exclusion criteria for ARM 1 were as follows:

1. Any subject with medical history of cancer except basal skin cancer or squamous skin cancer.

2. Any subject without PSA result or with PSA not within approved timeframe of at most 12 months.

3. Any subject who has had a DRE, ejaculated, or undertaken vigorous bike riding within 72 hours of blood draw.

4. Any subject with other lower urinary tract manipulation (defined as urological surgery, including prostate biopsy) in the previous 6 weeks from blood draw.

5. Any subject with benign prostatic hyperplasia as defined by the investigators review.

6. Any subject taking Saw Palmetto was excluded unless there is a minimum wash out of 30 days since last dose.

7. Any subject taking Androgen Deprivation Therapy

8. Any subject taking Casadex is excluded unless there is a minimum wash out of 30 days since the last dose.

9. Any patient currently taking an experimental agent - placebo control or unknown agent

10. Any subject taking 5 alpha reductase inhibitors is excluded unless there is a minimum 6 weeks washout since the last dose of finasteride and a minimum of 6 months wash out since the last dose of Dutasteride.

11. Any subject confirmed by the investigator to currently be suffering from prostatitis, proctodynia, or urinary tract infection.

ARM 2 prostate cancer biopsy exclusion criteria were as follows:

1. Any subject with medical history of cancer other than prostate cancer except basal or squamous skin cancer.

2. Any subject without PSA result or with PSA not within approved timeframe of at most 12 months.

3. Any subject who has had a DRE, ejaculated, or undertaken vigorous bike riding within 72 hours of blood draw

4. Any subject with other lower urinary tract manipulation (defined as urological surgery, including prostate biopsy) in the previous 6 weeks from blood draw.

5. Any subject taking Saw Palmetto is excluded unless there is a minimum wash out of 30 days since the last dose.

6. Any subject taking Androgen Deprivation Therapy

7. Any subject taking Casadex is excluded unless there is a minimum wash out of 30 days since the last dose.

8. Any patient currently taking an experimental agent - placebo control or unknown agent.

9. Any subject taking 5 alpha reductase inhibitors is excluded unless there is a minimum of 6 weeks washout since the last dose of finasteride and a minimum of 6 months wash out since the last dose of Dutasteride.

10. Any subject confirmed by the investigator to currently be suffering from prostatitis, proctodynia or urinary tract infection.

Study patient characteristics are outlined in Tables 1 and 2 below.

TABLE 1

patient characteristics

| Characteristic | Non-CaP | CaP | P value (non-CaP vs. CaP) | Non-AgCaP | AgCaP | P value (Non-AgCaP vs. AgCaP) |
|---|---|---|---|---|---|---|
| Total samples | 148 (45%) | 184 (55%) | | 64 (35%) | 120 (65%) | |
| Age | | | 0.39 | | | <0.01 |
| Mean (SD) | 64.07 (7.72) | 65.09 (8.16) | | 62.48 (7.51) | 66.48 (8.18) | |
| Median (range) | 65 (40-82) | 65 (45-85) | | 62 (45-79) | 66 (48-85) | |
| >50 years, N (%) | 141 (95%) | 179 (97%) | | 61 (95%) | 118 (98%) | |
| BMI | | | 0.74 | | | 0.21 |
| Mean (SD) | 29.30 (4.76) | 29.71 (6.57) | | 30.34 (6.65) | 29.37 (6.35) | |
| Median (range) | 28.69 (19.89-43.74) | 28.42 (17.90—72.55) | | 29.14 (21.28—60.22) | 27.93 (17.90- 72.55) | |
| Prostate volume (ml) | | | <0.01 | | | 0.02 |
| N measured (%) | 146 (99%) | 174 (95%) | | 58 (91%) | 116 (97%) | |
| Ultrasound | 57 | 61 | | 24 | 37 | |
| MRI | 7 | 17 | | 4 | 13 | |
| Mean (SD) | 64.03 (34.99) | 42.19 (19.36) | | 46.23 (18.40) | 40.17 (19.59) | |
| Median (range) | 52.40 (15.30—189.00) | 37.95 (12.70-121.30) | | 40.15 (18.10-94.80.) | 37.00 (12.70-121.30) | |
| Enrolling PSA (ng/ml) | | | <0.01 | | | <0.01 |
| Mean (SD) | 6.49 (3.84) | 9.77 (18.32) | | 6.25 (3.10) | 11.64 (22.38) | |
| Median (range) | 5.41 (1.2-31.73) | 6.31 (2-229) | | 5.63 (2-18.63) | 7.14 (3.65-229) | |

TABLE 1

patient characteristics (Continued)

| Characteristic | Non-CaP | CaP | P value (non-CaP vs. CaP) | Non-AgCaP | AgCaP | P value (Non-AgCaP vs. AgCaP) |
|---|---|---|---|---|---|---|
| Race, N (%) | | | 0.74 | | | 0.33 |
| White | 129 (87%) | 162 (88%) | | 54 (84%) | 108 (90%) | |
| Black | 17(11%) | 21 (11%) | | 10 (16%) | 11 (9%) | |
| Other/unknown | 2 (1%) | 1 (1%) | | 0 (0%) | 1 (1%) | |
| Hispanic Ethnicity,N (%) | | | 0.54 | | | 1.00 |
| Yes | 15 (10%) | 14 (8%) | | 5 (8%) | 9 (8%) | |
| No | 132 (89%) | 1.67 (91%) | | 58 (91%) | 109 (91%) | |
| Unknown | 1 (1%) | 3 (2%) | | 1 (2%) | 2 (2%) | |
| First deg. family history, N (%) | | | 0.20 | | | 0.20 |
| Yes | 33 (22%) | 57 (31%) | | 25 (39%) | 32 (27%) | |
| No | 98 (66%) | 110 (60%) | | 33 (52%) | 77 (64%) | |
| Unknown | 17 (11%) | 17 (9%) | | 6 (9%) | 11 (9%) | |
| DRE status | | | 0.02 | | | 0.03 |
| Normal | 115 (78%) | 119 (65%) | | 49 (77%) | 70 (58%) | |
| Suspicious | 15 (10%) | 39 (21%) | | 7 (11%) | 32 (27%) | |
| Unknown | 18 (12%) | 26 (14%) | | 8 (15%) | 18 (15%) | |
| Gleason Score/Epstein. N (%) | | | | | | |
| 6/1 | | 64 (35%) | | 64(100%) | | |
| 7 (3+4)/2 | | 58 (32%) | | | 58 (48%) | |
| 7 (4+3)/3 | | 43 (23%) | | | 43 (36%) | |
| 8/4 | | 5 (3%) | | | 5 (4%) | |
| 9/5 | | 14 (8%) | | | 14 (12%) | |

BMI = body mass index: CaP = prostate cancer; PSA = prostate specific antigen SD = standard deviation.
Continuous variables: Mann-Whitney.
Categorical variables: chi Square.

TABLE 2

Analysis of biomarker levels in non-CaP, CaP, aggressive and non-aggressive prostate cancer

| PSA, free PSA, proPSA and PHI | Non-Cap | CaP | P value | Non-AgCaP | AgCaP | P value |
|---|---|---|---|---|---|---|
| Total patients | 148 (45%) | 184(55%) | | 64 (35%) | 120(65%) | |
| Enrolling PSA (ng/ml) | | | <0.01 | | | <0.01 |
| Mean (SD) | 6.49 (3.84) | 9.77 (18.32) | | 6.25 (3.10) | 11.64 (22.38) | |
| Median (range) | 5.41 (1.2-31.73) | 6.31 (2-229) | | 5.63 (2-18.63) | 7.14(3.65-2.29) | |
| Central PSA (ng/ml) | | | <0.01 | | | <0.01 |
| Mean (SD) | 5.80 (3.01) | 10.39 (19.87) | | 5.79 (2.79) | 12.84 (14.19) | |
| Median (range) | 5.00 (1.20- 19.00) | 6.7 | | 5.60 (1.50-17.30) | 7.50 (2.40-236.60) | |
| <2 ng/ml, N (%) | 3 (2%) | 1 (1%) | <0.01 | 1 (2%) | 0 (0%) | |
| 2-10 ng/mL, N (%) | 135 (91%) | 142 (77%) | | 58 (91%) | 84 (70%) | |
| 4-10 ng/ml, N (%) | 100 (68%) | 121 (66%) | | 42 (66%) | 79 (66%) | |
| 3-15 ng/ml, N (%) | 127 (86%) | 154 (84%) | | 54 (34%) | 100 (83%) | |
| 10-20 ng/ml, N(%) | 11 (7%) | 31 (17%) | | 5 (8%) | 26 (22%) | |
| >20 ng/ml, N (%) | 0 (0%) | 10 (5%) | | 0 (0%) | 10 (8%) | |
| PHI | | | <0.01 | | | <0.01 |
| N measured (%) | 141 (95%) | 176 (96%) | | 62 (97%) | 114 (95%) | |
| Mean (SD) | 36.23 (16.26) | 60.81 (34.22) | | 43.9 (16.65) | 70 (37.71) | |

TABLE 2

Analysis of biomarker levels in non-CaP, CaP, aggressive and non-aggressive prostate cancer (Continued)

| PSA, free PSA, proPSA and PHI | Non-CaP | CaP | P value | Non-AgCaP | AgCaP | P value |
|---|---|---|---|---|---|---|
| Median (range) | 34.2 (9.7-149.5) | 52.35 (12.9-242.3) | | 43.95 (12.9-111.8) | 59.05(23.6-242.3) | |
| >50 yr + PSA 4-10 + Normal DRE | | | <0.01 | | | <0.01 |
| N measured (%) | 73 (49%) | 75 (41%) | | 32 (52%) | 43 (38%) | |
| Mean (SD) | 37.79 (12.50) | 53.81 (21.72) | | 44.88 (12.43) | 60.46 (24.72) | |
| Median (range) | 36 (15.9-74.3) | 49.7 (26.2-137.5) | | 45.4 (26.2-73.9) | 54.3(30-137.5) | |
| Total PSA (ng/ml) (PHI data) | | | <0.01 | | | <0.01 |
| N measured (%) | 141 | 178 | | 62 | 116 | |
| Mean (SD) | 4.84 (2.55) | 7.37 (8.73) | | 4.89 (2.36) | 8.7(10.45) | |
| Median (range) | 4.13 (1.04-15.18) | 5.42 (1.67-98.37) | | 4.67 (1.67-14.57) | 5.97 (2.62-98.37) | |
| Free PSA (ug/L) | | | 0.14 | | | 0.41 |
| N measured (%) | 141 (95%) | 176 (96%) | | 62 (97%) | 114(95%) | |
| Mean (SD) | 0.95 (0.57) | 0.87 (0.56) | | 0.83 (0.50) | 0.89(0.59) | |
| Median (range) | 0.85 (0.12-4.37) | 0.74 (0.18-4.46) | | 0.69 (0.18-2.65) | 0.77(0.22-4.46) | |
| %free PSA | | | <0.01 | | | <0.01 |
| N measured(%) | 141 (95%) | 176 (96%) | | 62 (97%) | 114(95%) | |
| Mean (SD) | 20.38 (8.89) | 14.47 (6.56) | | 17.64 (7.30) | 12.74 (5.42) | |
| Median (range) | 18.9 (5.5-62.1) | 12.9 (3.4-41.3) | | 16.65 (6.8-41.3) | 11.4 (3.4-28.2) | |
| proPSA (ng/L) | | | <0.01 | | | <0.01 |
| N measured (%) | 141 (95%) | 179 (97%) | | 62 (97%) | 117 (98%) | |
| Mean (SD) | 15.50 (10.94) | 27.73 (68.81) | | 16.12 (9.41) | 33.88 (84.31) | |
| Median (range) | 12.61 (3.29-70.41) | 16.45 (2.99-834.09) | | 13.64 (2.99-57.01) | 18.43 (3.24-834.09) | |

CaP = prostate cancer: PHI = prostate health index; PSA = prostate specific antigen; SD = standard deviation.

### 1.4 Sample Collection

Whole blood samples taken from patients were stored at 4° C. and subjected to centrifugation within 2 hours of collection to separate serum components, which were stored at -20° C. Samples were shipped from the collection sites then thawed, aliquoted, and stored at -80° C.

### 1.5 Multi-Analyte Arrays

Patient serum samples were thawed at room temperature then transferred to a 1.5 mL centrifuge tubes. The samples were spun at 20,000 g for 5 mins at room temperature. The middle fraction of each sample, avoiding any pellet or lipid layer, was transferred to 96-well plates and diluted with appropriate buffer. These sample plates were stored at -80° C. until they could be processed and run at the Australian Proteome Analysis Facility as per the manufacturer’s instructions. The samples were analyzed using a Bioplex 200 analyzer according to manufacturer’s instructions.

Two custom kits were obtained from R&D systems for this analysis:
The cytokines and growth factors contained in each kit were as follows:
29-plex: NT-proANP, Prolactin, ANGPTL3, Kallikrein 3.PSA, Endoglin, HGF, VEGF-C, CD31.Pecam1, Tie-2, SCF, VEGF R2.KDR.Flk-1, ErbB2.Her2, CXCL13.BLC.BCA-1, IL-7, FGF-b, HE4.WFDC-2, Angiopoietin-1, MADCAM-1, Leptin, BDNF, CD40 Ligand, IL-18, IL-6 R Alpha, uPA.Urokinase, PDGF-AB, Osteopontin, Mesothelin, EGF, CXCL12.SDF-1 alpha
3-plex: VEGF(VEGFA), G-CSF, Glypican-1

### 1.6 Model Development and Results

Samples from patients diagnosed with biopsy-confirmed prostate cancer from Arm 2 of the clinical trial were used for development of models differentiating aggressive (Gleason ≥3+4) from non-aggressive prostate cancer patients.

A combined database was generated linking the clinical and demographic factors to the analyte sample values. Following initial investigations, analyte concentrations derived from serum rather than plasma were used.
1. 332 clinical trial samples were measured using Minomic’s 29 and 3 Plex Luminex panels
2. Extreme haemolysed data (12 samples) were excluded, leading to 320 samples available for data analysis
3. Out of range and extrapolated data were set to either top or bottom values of standard curve for each analyte
4. PSA, %free PSA and HE4 analyte values were log transformed to achieve normal distribution for model development
5. No CaP: was defined as patients without prostate cancer (no cancer on biopsy)
6. CaP: patients with prostate cancer (GS ≥3+3)
7. NonAgCaP: patients with non-aggressive prostate cancer defined as Gleason Score equal to 3+3
8. NOT AgCaP = No CaP + NonAgCaP
9. AgCaP: patients with aggressive prostate cancer defined as biopsy Gleason Score equal to 3+4 or higher
10. 141 NoCaP, 62 NonAgCaP and 114 AgCaP samples had all data available for analysis (317 total)

These steps are summarized inthe flow chart below which indicates the breakdown of samples from the MiCheck-01 clinical trial used for analysis. [image] To develop multi-variate models, the following steps were used:

1. Imported the combined data set into the R² computer program loaded with the BMA³, VSURF^{4,5}, caret⁶, ROCR⁷, pROC⁸, stats packages.

2. Three clinical variables were mandated: PSA, DRE, %free PSA which are typically measured and commonly used in prostate cancer testing

3. Data from 22 of the 32 analytes measured using the 3-Plex and 29-Plex Luminex panels was used for analysis.
22 analytes: VEGF, G-CSF, Glypican-1, NT-proANP, Kallikrein 3, HGF, VEGF-C, Tie-2, VEGF R2/KDR/Flk-1, ErbB2/Her2, CXCL13.BLC.BCA-1, IL-7, WFDC2 (HE4), MADCAM-1, Leptin, CD40L, IL-18, IL.6.R.Alpha, uPA.Urokinase, PDGF.AB, osteopontin, mesothelin.

4. A stepwise regression was conducted using each of the analytes listed above: adding 1 marker into the mandated clinical factors to develop a model giving the best improvement in performance on both the CaP dataset or whole population. In particular, analytes increasing the specificity at a set 95% sensitivity were examined.

5. Result: WFDC2 (HE4) was identified as significantly contributing to an increase in specificity at 95% sensitivity in differentiating between non-AgCaP and AgCaP

Model development and ROC analyses (aggressive prostate cancer versus non-aggressive prostate cancer) were performed for PSA (FIG. One), DRE (FIG. Two), %free PSA (FIG. Three) and WFDC2 (HE4) (FIG. Four). The performance of the different models for the individual components is shown in Table 3.

TABLE 3

Performance of individual components in differentiating aggressive cancer from either non-aggressive cancer or non-aggressive and no cancer patients

| | AgCaP vs non-AgCaP 176 samples | | AgCaP vs NOT AgCaP 317 samples | |
|---|---|---|---|---|
| Component | AUC | P value | AUC | P value |
| PSA | 0.73 (0.65-0.81) | <0.001 | 0.73 (0.68-0.79) | <0.001 |
| DRE | 0.57 (0.51-0.63) | 0.043 | 0.57 (0.53-0.62) | 0.001 |
| %free PSA | 0.71 (0.64-0.79) | <0.001 | 0.76 (0.71-0.82) | <0.001 |
| HE4 | 0.61 (0.52-0.70) | 0.038 | 0.58 (0.5-0.65) | 0.009 |

The goal of the model development was to improve on currently available clinical tests such as PSA, DRE, or %free PSA the ability to accurately predict the presence of aggressive vs non-aggressive prostate cancer.

For each Logistic regression model, PSA, %free PSA and HE4 values were obtained and log transformed. The transformed values were multiplied by their respective log odds ratio co-efficient. If an abnormal/suspicious DRE status was obtained, it was multiplied by its log odds ratio co-efficient. The products were summed to generate a Logit(P) value which was then used in the following equation to generate a probability score P

The General equation is:

$$\begin{matrix}\text{Logit} \left(P\right) = \text{log} \left(P / 1 \text{-} P\right) = \text{intercept +} \sum \text{log} \text{odds} {\text{ratio}}_{i} \\ \times l o g \left(m a r k e {r}_{i}\right) + \sum \text{log} \text{odds} {\text{ratio}}_{D R E} \times 1 \left(\text{if} \text{suspicious} \text{DRE}\right) \\ P = \frac{e x p \left(L o g i t \left(P\right)\right)}{1 + e x p \left(L o g i t \left(P\right)\right)} \\ P \text{is} \text{a} \text{value} \text{between} \text{0} \text{and} \text{1} \text{that} \text{indicates} \text{the} \text{risk} \text{of} \text{AgCaP}\end{matrix}$$

Classification:
   If P > Threshold the patient is classified as having AgCaP

The contribution of additional analytes to the performance of different models is shown in Table 4.

TABLE 4

Comparison of models developed using 1-4 markers in the CaP and Whole evaluable population

| | | CaP population (176 samples - 114 AgCaP vs 62 NonAgCaP) | | CaP model applied to whole population (317 sample - 114 AgCaP vs 203 others) | |
|---|---|---|---|---|---|
| Model | Component(s) | AUC | Specificity at 95% sensitivity | AUC | Specificity at 95% sensitivity |
| (a) | PSA | 0.73 (0.65-0.81) | 29 | 0.73 (0.68 - 0.79) | 32 |
| (b) | DRE | 0.57 (0.51-0.63) | n/a | 0.57 (0.53-0.62) | n/a |
| (c) | %free PSA | 0.71 (0.61-0.79) | 16 | 0.76 (0.71-0.82 | 27 |
| (d) | WFDC2(HE4) | 0.61 (0.52-0.70) | 11 | 0.58 (0.52-0.65) | 16 |
| (e) | PSA, DRE | 0.76 (0.69 - 0.84) | 26 | 0.76 (0.71-0.81) | 32 |
| (f) | PSA, DRE, %free PSA | 0.80 (0.73-0.86) | 26 | 0.82 (0.77-0.87) | 33 |
| (g) | PSA, DRE, %freePSA, WFDC2(HE4) | 0.80 (0.73-0.87) | 40 | 0.83 (0.78-0.88) | 46 |

TABLE 5

Comparison of performance of models (f) and (g) in CaP and Whole evaluable population

| Comparison of Models (f) and (g) | CaP population | | CaP model applied to whole population | |
|---|---|---|---|---|
| Diffence in AUC | 0 | p value = 0.609 | 0.1 | p value = 0.077 |
| Difference in specificity at 95%Sens | 14% | p value = 0.003 | 13% | P value = 2.38e-05 |

Of note, addition of DRE to PSA increased the AUC in differentiating AgCaP from non-AgCaP in the CaP population (0.76 vs 0.73), while inclusion of %free PSA further increased the AUC (0.80 vs 0.76). Addition of WFDC2 (HE4) did not further improve the AUC in this population (Table 4). The specificity at 95% sensitivity was not improved by addition of DRE and %free PSA to PSA. However, inclusion of WFDC2 (HE4) significantly increased the specificity at 95% sensitivity in the CaP population (40% vs 26%, p = 0.003, Tables 4 and 5).

When the model (g) was applied to the whole population, inclusion of WFDC2 (HE4) increased the AUC compared to model (f) (0.83 vs 0.82, Table 4) but this did not reach statistical significance (p = 0.077, Table 5). Inclusion of WFDC2 (HE4) significantly increased the specificity at 95% sensitivity in this population (46% vs 33%, p = 2.38x10⁻⁵).

To further optimise the model development using the variables PSA, DRE, %free PSA, and WFDC2 (HE4), the following approach was adopted:
1. Model MiCheck Prostate 1a_{standard} was developed on the CaP population only, using standard multivariable logistic regression modelling
2. Model MiCheck Prostate 1b_{standard} was developed on the whole population, using standard multivariable logistic regression modelling
3. Performance was then assessed on the whole population using both models
4. Model MiCheck Prostate 1a_{standard} had better performance than Model MiCheck Prostate 1b_{standard} therefore, model MiCheck Prostate 1aᵥₐₗ was developed on the CaP population only, using cross-validated (“val”) multivariable logistic regression model; then applied to the whole population
5. Two versions of model MiCheck Prostate 1aᵥₐₗ were obtained following the cross-validation: V1 had slightly high specificity at 95% sensitivity on whole population while V2 was more balanced in both AUC and specificity at 95% sensitivity between training and test sets.

These steps are set out in more detail below.

### (a) standard logistic regression model 1a developed on the cap population only

Model developed to differentiate AgCaP vs NonAgCaP in CaP population
Data for model development: CaP patients only (114 AgCaP, 62 NonAgCaP)
Data for performance report: CaP patients only (114 AgCaP, 62 NonAgCaP)
Method: Standard Multivariable Logistic Regression
AUC is 0.8 (0.73-0.87), ROC curve is shown in FIG. Five

TABLE 6A

| Variable | Transformation | Log Odd ratio |
|---|---|---|
| (Intercept) | | -5.27815948514258 |
| Central.PSA | Log | 1.57489770949082 |
| Abnormal DRE | | 1.11429816720971 |
| %Free.PSA | Log | -1.5306904330285 |
| WFDC2 (HE4) | Log | 0.763176752224671 |

TABLE 6B

| Sensitivity (%) | Specificity (%) | Accuracy (%) |
|---|---|---|
| 90 | 50 | 75.6 |
| 95 | 40 | 75.6 |

### (B) Standard Logistic Regression Model 1a Applied to the Whole Patient Population

The model developed in (a) was applied to the whole patient population.
Data for model development: CaP patients only (114 AgCaP, 62 NonAgCaP)
Data for performance report: whole evaluable population (114 AgCaP, 203 NOTAg CaP)
Method: Standard Multivariable Logistic Regression
AUC is 0.83 (0.78-0.88), ROC curve is shown in FIG. Six

### (C) Assessment Of Micheck 1a Test Performance On Whole Population

When applied to the whole population using a cutpoint of 95% sensitivity, The MiCheck 1a_{standard} algorithm classifies 218 patients as positive and 99 patients as negative. The breakdown of test results and the NPV for GS≥3+4 and GS≥4+3 are shown below in Table 7. The percentage reduction in biopsies for no CaP, NonAgCaP and AgCaP are shown in FIG. Seven.

TABLE 7

Algorithm outcomes for MiCheck1a_{standard} applied to the whole patient population
Algorithm Outcomes

| Diagnosis | Of 218 positive MiCheck tests | Of 99 negative MiCheck tests |
|---|---|---|
| No Cancer | 73 | 68 |
| Non Aggressive CaP | 37 | 25 |
| Aggressive CaP | 108 | 6 (5 GS 3+4, 1 GS 4+3 0 GS >4+3) |

46% of unnecessary biopsies saved
NPV (GS ≥3+4) = 93.9%
NPV (GS ≥4+3) = 99%
5% GS ≥3+4 cancers delayed diagnosis
1.8% GS ≥4+3 cancers delayed diagnosis
0% GS ≥8 cancers delayed diagnosis

### (D) Standard Regression Model 1b Developed on Whole Patient Population

Model developed to differentiate AgCaP vs NOT AgCaP in whole population
Data for model development: whole study population (114 AgCaP, 203 NOT AgCaP)
Data for performance report: whole study population (114 AgCaP, 203 NOT AgCaP)
Method: Standard Multivariable Logistic Regression
AUC is 0.83 (0.78-0.88), ROC Curve is shown in FIG. Eight

TABLE 8A

| Variable | Transformation | Log Odd ratio |
|---|---|---|
| (Intercept) | | -5.34746217622658 |
| Central.PSA | Log | 1.36753205476678 |
| Abnormal DRE | | 1.07370376051641 |
| %Free.PSA | Log | -2.23325453386807 |
| WFDC2 (HE4) | Log | 0.903522236886068 |

TABLE 8B

| Sensitivity (%) | Specificity (%) | Accuracy (%) |
|---|---|---|
| 90 | 53 | 65.9 |
| 95 | 35 | 56.8 |

### (E) Assessment Of Micheck 1b_{Standard} Test Performance On Whole Population

When applied to the whole population using a cutpoint of 95% sensitivity, The MiCheck 1b_{standard} algorithm classifies 239 patients as positive and 78 patients as negative. The breakdown of test results and the NPV for GS≥3+4 and GS≥4+3 are shown below in Table 9. The percentage reduction in biopsies for no CaP, non-AgCaP and AgCaP are shown in FIG. Nine.

TABLE 9

Algorithm outcomes for MiCheck1b applied to the whole patient population
Algorithm Outcomes

| Diagnosis | Of 239 positive MiCheck tests | Of 78 negative MiCheck tests |
|---|---|---|
| No Cancer | 88 | 53 |
| Non Aggressive CaP | 43 | 19 |
| Aggressive CaP | 108 | 6 (5 GS 3+4, 1 GS 4+3 0 GS>4+3) |

35% of unnecessary biopsies saved
NPV (≥3+4) = 92%
NPV (≥4+3) = 99%
5% GS ≥3+4 cancers delayed diagnosis
1.8% GS ≥4+3 cancers delayed diagnosis
0% GS ≥8 cancers delayed diagnosis

### (F) Comparison of Standard Logistic Regression Model Performance

TABLE 10

Comparison of Models 1a and 1b

| Model | Performance on whole evaluable MiCheck-01 Prostate population (%) | | | | | | | | | | |
|---|---|---|---|---|---|---|---|---|---|---|---|
| | AUC | Sens | Spec (Unnecessary biopsies saved) | Acc | Total biopsies saved* | PPV | NPV (≥3+4) | NPV (≥4+3) | Delayed GS≥3+4 | Delayed GS≥4+3 | Delayed GS≥8 |
| MiCheck® Prostate 1a | 0.83 (0.78-0.88) | 95 | 46 | 63.4 | 31 | 50 | 94 | 99 | 5 | 1.8 | 0 |
| MiCheck® Prostate 1b | 0.83 (0.78-0.88) | 95 | 35 | 56.8 | 25 | 45 | 92 | 99 | 5 | 1.8 | 0 |

*If a MiCheck® Prostate test is negative then biopsies would not be performed in these cases

Model MiCheck 1a was developed on the CaP population only, then applied to the whole population to determine its performance characteristics
Model MiCheck 1b was developed on the whole population, then applied to the whole population to determine its performance characteristics
The test performance at the clinicians desired sensitivity of 95% sensitivity for aggressive cancer was compared
Model MiCheck 1a has superior specificity (46% vs 35%) at 95% sensitivity and thus higher unnecessary biopsies saved, as well as a higher % total biopsies saved (31% vs 25%) with equivalent delayed detection of aggressive CaP when compared to Model MiCheck 1b

### (G) Development Of Cross-Validated Models Using Cap Population

As Model 1a had proved superior to Model 1b, the CaP population was used for development of cross-validated models. Monte Carlo cross-validation was applied to avoid overfitting. The data was split into two thirds for training and one third for test, repeated 2000 times. The proportion of Non-AgCaP to AgCaP in the training and test data sets was equivalent and is shown in FIG. Ten. For each split, a multivariable logistic regression model consisting of 4 variables was developed using the training data set. The model was then compared in the complementary test data set to get the performance. Several models with the same optimal performance were obtained, thus additional performance criteria were applied such that the final model and cutpoint should permit no more than 5% of AgCaP having GS 4+3 and no Gleason 8 or higher cancers to be classified as negative, while maximizing biopsies saved. The process is shown in the schematic below outlining the cross-validation process using training and test data sets.

[image]

Following the cross-validation process, two models were selected. The relative performance in the training and test datasets, together with the whole population is shown in the schematic below, which shows a summary of test performance of the top two models derived from the Monte-Carlo cross-validation process, while a comparison of both models with Model 1a_{standard} is shown in Table 11.

[image]

TABLE 11

Comparison of Model 1a_{standard,} V1 and V2 cross validated models on the whole patient population

| Model | Performance on whole evaluable MiCheck-01 Prostate population (%) | | | | | | | | | | |
|---|---|---|---|---|---|---|---|---|---|---|---|
| | AUC | Sens | Spec (Unnecessary biopsies saved) | Acc | Total biopsies saved* | PPV | NPV (≥3+4) | NPV (≥4+3) | Delayed GS≥3+4 | Delayed GS≥4+3 | Delayed GS≥8 |
| MiCheck® Prostate 1a_{standard} | 0.83 (0.78-0.88) | 95 | 46 | 63.4 | 31 | 50 | 94 | 99 | 5 | 1.8 | 0 |
| V1-MiCheck® Prostateᵥₐₗ | 0.82 (0.77-0.87) | 95 | 48 | 64.7 | 33 | 50 | 94 | 99 | 5 | 1.8 | 0 |
| V2-MiCheck® Prostateᵥₐₗ | 0.83 (0.78-0.88) | 95 | 47 | 64.0 | 32 | 50 | 94 | 99 | 5 | 1.8 | 0 |

3 models were developed on the CaP population only, then applied to the whole population to determine their performance characteristics
Model MiCheck Prostate 1a_{standard} was developed using standard multivariable logistic regression;
V1-MiCheck Prostateᵥₐₗ and V2-MiCheck Prostateᵥₐₗ were developed using cross-validation multiple logistic regression
V1-MiCheck Prostateᵥₐₗ has superior specificity and thus unnecessary biopsies saved (48% vs 46%) and %total biopsies saved (33% vs 31%) with equivalent delayed detection of aggressive CaP when compared to Model MiCheck Prostate 1a_{standard}
V1-MiCheck Prostateᵥₐₗ had slightly higher specificity at 95% sensitivity on the whole population compared to V2 (48% vs 47%), however V2-MiCheck® Prostateᵥₐₗ was more balanced in both AUC and specificity at 95% sensitivity between training and test sets.

### (H) V1 Micheck 1a_{Validated} Cross-Validated Models On Cap Patient Population

Model developed to differentiate AgCaP vs NonAgCaP in CaP population
Data for model development: CaP patients only (114 AgCaP, 62 NonAgCaP)
Data for model performance: CaP patients only (114 AgCaP, 62 NonAgCaP)
Method: cross-validated standard Multivariable Logistic Regression
AUC is 0.8 (0.73-0.87), ROC Curve is shown in FIG. Eleven

TABLE 12A

| Variable | Transformation | Log Odd ratio |
|---|---|---|
| (Intercept) | | -5.21589841264147 |
| Central.PSA | Log | 1.81345525269023 |
| Abnormal DRE | | 0.726194851146861 |
| %Free.PSA | Log | -1.33080567063805 |
| WFDC2 (HE4) | Log | 0.641871684205315 |

TABLE 12B

| Sensitivity (%) | Specificity (%) | Accuracy (%) |
|---|---|---|
| 90 | 53 | 76.1 |
| 95 | 44 | 76.7 |

### (I) V1 Micheck 1a_{Validated} Cross-Validated Model On Whole Patient Population

Model developed to differentiate AgCaP vs NonAgCaP in CaP population
Data for model development: CaP patients only (76 AgCaP, 42 NonAgCaP)
Data for model performance: CaP patients only (114 AgCaP, 203 NOTAg CaP)
Method: cross-validated standard Multivariable Logistic Regression
AUC is 0.82 (0.77-0.87), ROC Curve is shown in FIG. Twelve

TABLE 13

| Sensitivity (%) | Specificity (%) | Accuracy (%) |
|---|---|---|
| 90 | 54 | 66.9 |
| 95 | 48 | 64.7 |

### (J) Assessment Of V1 Micheck 1a_{Validated} Cross-Validated Model On Whole Patient Population

When applied to the whole population using a cutpoint of 95% sensitivity, The V1 MiCheck 1a_{validated} algorithm classifies 214 patients as positive and 103 patients as negative. The breakdown of test results and the NPV for GS≥3+4 and GS≥4+3 are shown below in Table 14. The percentage reduction in biopsies for no CaP, non-AgCaP and AgCaP are shown in FIG. Thirteen.

TABLE 14

Performance of V1 MiCheck1a_{validated} on whole patient population
Algorithm Outcomes

| Diagnosis | Of 214 positive MiCheck tests | Of 103 negative MiCheck tests |
|---|---|---|
| No Cancer | 71 | 70 |
| Non Aggressive CaP | 35 | 27 |
| Aggressive CaP | 108 | 6 (5 GS 3+4, 1 GS 4+3 0 GS>4+3) |

48% of unnecessary biopsies saved
NPV (GS≥3+4) = 94.2%
NPV (GS≥4+3) = 99.0%
5.3% GS ≥3+4 cancers delayed diagnosis
1.8% GS ≥4+3 cancers delayed diagnosis
0% GS≥8 cancers delayed diagnosis

### (K) V2 Micheck 1a_{Validated} Cross-Validated Models On Cap Patient Population

Model developed to differentiate AgCaP vs NonAgCaP in CaP population
Data for model development: CaP patients only (114 AgCaP, 62 NonAgCaP)
Data for model performance: CaP patients only (114 AgCaP, 62 NonAgCaP)
Method: cross-validated standard Multivariable Logistic Regression
AUC is 0.8 (0.73-0.87), ROC Curve is shown in FIG. Fourteen

TABLE 15A

| Variable | Transformation | Log Odd ratio |
|---|---|---|
| (Intercept) | | -2.941061748 |
| Central.PSA | Log | 1.6660440801736 |
| Abnormal DRE | | 1.16657333364167 |
| %Free.PSA | Log | -1.72680200527853 |
| WFDC2 (HE4) | Log | 0.537737024994997 |

TABLE 15B

| Sensitivity (%) | Specificity (%) | Accuracy (%) |
|---|---|---|
| 90 | 45 | 74.4 |
| 95 | 37 | 74.4 |

### (L) V2 Micheck 1a_{Validated} Cross-Validated Model On Whole Patient Population

Model developed to differentiate AgCaP vs NonAgCaP in CaP population
Data for model development: CaP patients only (176 AgCaP, 42 NonAgCaP)
Data for model performance: CaP patients only (114 AgCaP, 203 NOTAg CaP)
Method: cross-validated standard Multivariable Logistic Regression
AUC is 0.83 (0.78-0.88), ROC Curve is shown in FIG. Fifteen

TABLE 16

| Sensitivity (%) | Specificity (%) | Accuracy (%) |
|---|---|---|
| 90 | 52 | 65.6 |
| 95 | 47 | 64.0 |

### (M) Assessment Of V2 Micheck 1a_{Validated} Cross-Validated Model On Whole Patient Population

When applied to the whole population using a cutpoint of 95% sensitivity, The V2 MiCheck 1a_{validated} algorithm classifies 216 patients as positive and 101 patients as negative. The breakdown of test results and the NPV for GS≥3+4 and GS≥4+3 are shown below in Table 17. The percentage reduction in biopsies for no CaP, non-AgCaP and AgCaP are shown in FIG. Sixteen.

TABLE 17

Performance of V2 MiCheck1a_{validated} on whole patient population
Algorithm Outcomes

| Diagnosis | Of 216 positive MiCheck tests | Of 101 negative MiCheck tests |
|---|---|---|
| No Cancer | 69 | 72 |
| Non Aggressive CaP | 39 | 23 |
| Aggressive CaP | 108 | 6 (5 GS 3+4, 1 GS 4+3 0 GS >4+3) |

47% of unnecessary biopsies saved
NPV (GS≥3+4) = 94.1%
NPV (GS≥4+3) = 99.0%
5.3% GS ≥3+4 cancers delayed diagnosis
1.8% GS ≥4+3 cancers delayed diagnosis
0% GS≥8 cancers delayed diagnosis

### (N) Assessment of V1 MiCheck 1a_{validated} Cross-Validated Model on Patient Population PSA Range 2-10 ng/ml and PSA 4-10 ng/ml

There is ongoing debate about the optimum PSA value to use as a threshold for biopsy. A PSA value of >4 ng/ml has been historically used as a threshold for biopsy, while others have proposed >3 ng/ml or even lower at >1.5 ng/ml⁹. The PSA “grey zone” of 4-10 ng /ml is particularly problematic as only 26% of patients have prostate cancer.

The V1 MiCheck 1a_{validated} model was tested in patients in the PSA range of 2-10 ng/ml and 4-10 ng/ml using the same cutpoint that gives 95% sensitivity in the whole evaluable PSA range population.

The test performance in these groups is shown below in Table 18.

TABLE 18

Performance of V1 MiCheck1a_{validated} on whole different PSA ranges
Performance of models in different PSA ranges
A. Whole PSA range

| Model | | Variables | | | | AUC (95%CI) | | | Sens (%) | Spec (%) | Acc (%) | %Biosy saved |
|---|---|---|---|---|---|---|---|---|---|---|---|---|
| V1-MiCheck® Prostate-val | | DRE, PSA. %Free PSA, HE4 | | | | 0.82 (0.77-0.87) | | | 95 | 48 | 65 | 33 |
| B. PSA range 2-10 ng/ml | | | | | | | | | | | | |
| V1-MiCheck® Prostale-val | | DRE, PSA. %Free PSA, HE4 | | | | 0.80 (0.74-0.86) | | | 93 | 51 | 64 | 38 |
| C. PSA range 4-10 ng/ml | | | | | | | | | | | | |
| V1-MiCheck® Prostate-val | | DRE, PSA, %Free PSA, HE4 | | | | 0.78 (0.71-0.84) | | | 96 | 36 | 58 | 24.8 |
| A. Whole PSA range | | | | | | | | | | | | |
| | PPV | | NPV GS≥3+4 | NPV GS≥4+3 | Delayed GS≥3+4 | | Delayed GS≥4+3 | Delayed GS≥8 | Sample sizes (No CaP/Non AgCap/AgCaP) | | | |
| | 50 | | 94 | 99 | 5 | | 1.8 | 0 | 141/62/114 | | | |
| B. PSA range 2-10 ng/ml | | | | | | | | | | | | |
| | 46 | | 94 | 99 | 7.3 | | 2.5 | 0 | 128/57/82 | | | |
| C. PSA range 4- 10 ng/ml | | | | | | | | | | | | |
| | 46 | | 94.3 | 98.1 | 3.9 | | 2.6 | 0 | 96/41/77 | | | |

### (O) Development of Models with Prostate Volume

Prostate volume is often collected during MRI assessment of patients with suspected prostate cancer. Prostate volume was significantly higher in no cancer and non-aggressive cancer patients than in aggressive prostate cancer patients (see Table 19). Prostate volume was therefore incorporated into the variables for model development, either as a substitute for DRE or together with DRE.

Prostate volume was collected for 110 AgCaP, 56 Non-AgCaP and 139 NoCaP subjects. Individual analyte AUCs and p values for differentiating non-aggressive cancer or non-aggressive and no cancer patients are shown in Table 19.

TABLE 19

Performance of individual components in differentiating aggressive cancer from either non-aggressive cancer or non-aggressive and no cancer patients in patient subset with PV data

| | AgCaP vs non-AgCaP 166 samples | | AgCaP vs NOT AgCaP 305 samples | |
|---|---|---|---|---|
| Component | AUC | P value | AUC | P value |
| PSA | 0.73 (0.66-0.81) | 0.0002 | 0.73 (0.68-0.79) | <0.0001 |
| DRE | 0.58 (0.52-0.64) | 0.024 | 0.58 (0.53-0.62) | 0.0006 |
| PV | 0.62 (0.53-0.71) | 0.041 | 0.70 (0.64-0.76) | <0.0001 |
| %free PSA | 0.70 (0.61-0.78) | <0.0001 | 0.76 (0.70-0.81) | <0.0001 |
| HE4 | 0.62 (0.53-0.71) | 0.127 | 0.59 (0.53-0.66) | 0.090 |

The goal of the model development was to improve on currently available clinical tests such as PSA, DRE, PV or %free PSA the ability to accurately predict the presence of aggressive vs non-aggressive prostate cancer.

For each Logistic regression model, PSA, %free PSA, PV and HE4 values were obtained and log transformed. The transformed values were multiplied by their respective log odds ratio co-efficient. If an abnormal/suspicious DRE status was obtained, it was multiplied by its log odds ratio co-efficient. The products were summed to generate a Logit(P) value which was then used in the following equation to generate a probability score P

The General equation is:

$$\begin{matrix}\text{Logit} \left(P\right) = \text{log} \left(P / 1 \text{-} P\right) = \text{intercept} \text{+} \sum \text{log} \text{odds} {\text{ratio}}_{i} \\ \times l o g \left(m a r k e {r}_{i}\right) \\ P = \frac{e x p \left(L o g i t \left(P\right)\right)}{1 + e x p \left(L o g i t \left(P\right)\right)} \\ P \text{is} \text{a} \text{value} \text{between} \text{0} \text{and} \text{1} \text{that} \text{indicates} \text{} \text{the} \text{risk} \text{of} \text{AgCaP}\end{matrix}$$

Classification:
   If P > Threshold the patient is classified as having AgCaP

The contribution of additional analytes to the performance of different models is shown in Table 20.

TABLE 20

Comparison of models developed using 1-4 markers in the CaP and Whole evaluable population

| | | CaP population (166 samples - 110 AgCaP vs 56 NonAgCaP) | | CaP model applied to whole population (305 sample - 110 AgCaP vs 195 others) | |
|---|---|---|---|---|---|
| Model | Component(s) | AUC (95%CI) | Specificity at 95%Sens | AUC (95%CI) | Specificity at 95% Sensitivity |
| (a) | PSA | 0.73 (0.66 - 0.81) | 27 | 0.73 (0.68 - 0.79) | 31 |
| (b) | DRE | 0.58 (0.52 - 0.64) | n/a | 0.58 (0.53 - 0.62) | n/a |
| (c) | PV | 0.62 (0.53 - 0.71) | 10 | 0.70 (0.64 - 0.76) | 22 |
| (d) | %free PSA | 0.70 (0.61 - 0.78) | 14 | 0.76 (0.70 - 0.81) | 27 |
| (e) | HE4 | 0.62 (0.53 - 0.72) | 13 | 0.59 (0.53 - 0.66) | 16 |
| (f) | PSA, PV | 0.77 (0.70 - 0.84) | 29 | 0.82 (0.77-0.87) | 44 |
| (g) | PSA, PV, %free PSA | 0.78 (0.71 - 0.85) | 29 | 0.83 (0.78 - 0.88) | 39 |
| (h) | PSA, PV, %freePSA, HE4 | 0.80 (0.73 - 0.87) | 36 | 0.85 (0.80 - 0.89) | 45 |

TABLE 21

Comparison of performance of models (g) and (h) in CaP and Whole evaluable population

| Comparison of Models (g) and (h) | CaP population (166 samples - 110 AgCaP vs 56 NonAgCaP) | | CaP model applied to whole population (305 sample - 110 AgCaP vs 195 others) | |
|---|---|---|---|---|
| Difference in AUC | 0.02 | p value = 0.355 | 0.02 | p value = 0.355 |
| Difference in specificity at 95%Sens | 7% | p value = 0.289 | 6% | P value = 0.090 |

Of note, addition of PV to PSA increased the AUC in differentiating AgCaP from non-AgCaP in the CaP population (0.77 vs 0.73), while inclusion of %free PSA resulted in a minor further increase in the AUC (0.78 vs 0.77). Addition of WFDC2 (HE4) resulted in further improve the AUC in this population (0.80 vs 0.78, Table 20). The specificity at 95% sensitivity showed a small increase following addition of PV and %free PSA to PSA. However, inclusion of WFDC2 (HE4) resulted in increased specificity at 95% sensitivity in the CaP population (36% vs 29%) however this did not reach statistical significance (p = 0.289, Table 21).

When the model (h) was applied to the whole population, inclusion of WFDC2 (HE4) increased the AUC compared to model (g) (0.85 vs 0.83, Table 20) but this did not reach statistical significance (p = 0.355, Table 21). Inclusion of WFDC2 (HE4) increased the specificity at 95% sensitivity in this population (45% vs 39%) but this did not achieve statistical significance (p=0.09, Table 21).

To further optimise the model development using the variables PSA, PV, %free PSA, and WFDC2 (HE4), the following approach was adopted:
1. Model MiCheck Prostate 1a_{standardPV} was developed on the CaP population only, using standard multivariable logistic regression modelling
2. Model MiCheck Prostate 1b_{standardPV} was developed on the whole population, using standard multivariable logistic regression modelling
3. Performance was then assessed on the whole population using both models
4. Model MiCheck Prostate 1a_{standardPV} had better performance than Model MiCheck Prostate 1b_{standardPV} therefore, model MiCheck Prostate 1aᵥₐₗ was developed on the CaP population only, using cross-validated (“val”) multivariable logistic regression model; then applied to the whole population
5. An optimal version of model MiCheck Prostate 1a_{valPV} was obtained following the cross-validation.

These steps are set out in more detail below.

### (P) Standard Logistic Regression Model 1a_{PV} Developed on the CaP Population Only

Model developed to differentiate AgCaP vs NonAgCaP in CaP population
Data for model development: CaP patients only (110 AgCaP, 56 NonAgCaP)
Data for performance report: CaP patients only (110 AgCaP, 56 NonAgCaP)
Method: Standard Multivariable Logistic Regression
AUC is 0.8 (0.73-0.87), ROC curve is shown in FIG. Seventeen

TABLE 22A

| Variable | Transformation | Log Odd ratio |
|---|---|---|
| (Intercept) | | -7.28432071327325 |
| Central.PSA | Log | 1.68497375260022 |
| Prostate Volume | Log | -0.86924621606277 |
| %Free.PSA | Log | -0.91791135785732 |
| WFDC2 (HE4) | Log | 1.16906804572333 |

TABLE 22B

| Sensitivity (%) | Specificity (%) | Accuracy (%) |
|---|---|---|
| 90 | 43 | 74.1 |
| 95 | 36 | 74.7 |

### (Q) Standard Logistic Regression Model 1a_{PV} Applied to the Whole Patient Population

The model developed in (a) was applied to the whole patient population.
Data for model development: CaP patients only (110 AgCaP, 56 NonAgCaP)
Data for performance report: whole evaluable population (110 AgCaP, 195 NOT AgCaP)
Method: Standard Multivariable Logistic Regression
AUC is 0.85 (0.80-0.89), ROC curve is shown in FIG. Eighteen

### (R) Assessment Of Micheck 1a_{Pv} Test Performance On Whole Population

When applied to the whole population with available PV data using a cutpoint of 95% sensitivity, The MiCheck 1a_{standardPV} algorithm classifies 211 patients as positive and 94 patients as negative. The breakdown of test results and the NPV for GS≥3+4 and GS≥4+3 are shown below in Table 23. The percentage reduction in biopsies for no CaP, NonAgCaP and AgCaP are shown in FIG. Nineteen.

TABLE 23

Algorithm outcomes for MiCheck1a_{standardPV} applied to the whole patient population
Algorithm Outcomes

| Diagnosis | Of 211 positive MiCheck® tests | Of 94 negative MiCheck® tests |
|---|---|---|
| No Cancer | 71 | 68 |
| Non Aggressive CaP | 36 | 20 |
| Aggressive CaP | 104 | 6 (5 GS 3+4, 1 GS 4+3 0 GS>4+3) |

45% of unnecessary biopsies saved
NPV (≥3+4) = 93.6%
NPV (≥4+3) = 98.9%
5% GS ≥3+4 cancers delayed diagnosis
1% GS ≥4+3 cancers delayed diagnosis
0% GS ≥8 cancers delayed diagnosis

### (S) Standard Logistic Regression Model 1b_{Pv} Developed On Whole Patient Population

Model developed to differentiate AgCaP vs NOT AgCaP in whole population
Data for model development: whole study population (110 AgCaP, 195 NOT AgCaP)
Data for performance report: whole study population (110 AgCaP, 195 NOT AgCaP)
Method: Standard Multivariable Logistic Regression
AUC is 0.84 (0.79-0.89), ROC Curve is shown in FIG. Twenty

TABLE 24A

| Variable | Transformation | Log Odd ratio |
|---|---|---|
| (Intercept) | | -0.18132 |
| Central.PSA | Log | 0.223112 |
| Prostate Volume | Log | -0.03075 |
| %Free.PSA | Log | -0.08469 |
| WFDC2 (HE4) | Log | 0.000107 |

TABLE 24B

| Sensitivity (%) | Specificity (%) | Accuracy (%) |
|---|---|---|
| 90 | 47 | 62.6 |
| 95 | 36 | 53.4 |

### (T) Assessment Of Micheck 1b_{Standardpv} Test Performance On Whole Population

When applied to the whole population using a cutpoint of 95% sensitivity, The MiCheck 1b_{standardPV} algorithm classifies 228 patients as positive and 77 patients as negative. The breakdown of test results and the NPV for GS≥3+4 and GS≥4+3 are shown below in Table 25. The percentage reduction in biopsies for no CaP, non-AgCaP and AgCaP are shown in FIG. Twenty One.

TABLE 25

Algorithm outcomes for MiCheck1bₚᵥ applied to the whole patient population.
Algorithm Outcomes

| Diagnosis | Of 228 positive MiCheck tests | Of 77 negative MiCheck tests |
|---|---|---|
| No Cancer | 78 | 61 |
| Non Aggressive CaP | 46 | 10 |
| Aggressive CaP | 104 | 6 (4 GS 3+4, 2 GS 4+3 0 GS>4+3) |

36% of unnecessary biopsies saved
NPV (≥3+4) = 92.2%
NPV (≥4+3) = 97.4%
5.5% GS ≥3+4 cancers delayed diagnosis
1.8% GS ≥4+3 cancers delayed diagnosis
0% GS ≥8 cancers delayed diagnosis

### (U) Comparison of Standard Logistic Regression Model Performance

Model MiCheck 1aₚᵥ was developed on the CaP population only, then applied to the whole population to determine its performance characteristics
Model MiCheck 1bₚᵥ was developed on the whole population, then applied to the whole population to determine its performance characteristics
The test performance at the clinicians desired sensitivity of 95% sensitivity for aggressive cancer was compared
Model MiCheck 1aₚᵥ has superior specificity (45% vs 36%) at 95% sensitivity and thus higher unnecessary biopsies saved, when compared to Model MiCheck 1ₚᵥ

### (V) Development Of Cross-Validated Models Using Cap Population

As Model 1aₚᵥ had proved superior to Model 1bₚᵥ, the CaP population was used for development of cross-validated models. Monte Carlo cross-validation was applied to avoid overfitting. The data was split into two thirds for training and one third for test, repeated 2000 times. The proportion of Non-AgCaP to AgCaP in the training and test data sets was equivalent and is shown in FIG. Twenty Two. For each split, a multivariable logistic regression model consisting of 4 variables was developed using the training data set. The model was then compared in the complementary test data set to get the performance. Several models with the same optimal performance were obtained, thus additional performance criteria were applied such that the final model and cutpoint should permit no more than 5% of AgCaP having GS 4+3 and no Gleason 8 or higher cancers to be classified as negative, while maximizing biopsies saved. A schematic of the process is shown below. [image] Following the cross-validation process, an optimal model was selected. The ROC curves for the training and test datasets are shown in FIGS. Twenty Three and Twenty Four respectively. The ROC curve for performance in the whole evaluable CaP population is shown in FIG. Twenty Five while the performance in the whole population is shown in FIG. Twenty Six.

### (W) Micheck 1a_{Validatedpv} Cross-Validated Models On Cap Patient Population

Model developed to differentiate AgCaP vs NonAgCaP in CaP population
Data for model development: CaP patients only (74 AgCaP, 38 NonAgCaP)
Data for model performance: CaP patients only (110 AgCaP, 56 NonAgCaP)
Method: cross-validated standard Multivariable Logistic Regression
AUC is 0.8 (0.73-0.87), ROC Curve is shown in FIG. Twenty Five

TABLE 26A

| Variable | Transformation | Log Odd ratio |
|---|---|---|
| (Intercept) | | -6.987790281 |
| Central.PSA | Log | 1.60588637 |
| Prostate Volume | Log | -0.677092452 |
| %Free.PSA | Log | -0.956208098 |
| WFDC2 (HE4) | Log | 1.078503801 |

TABLE 26B

| Sensitivity (%) | Specificity (%) | Accuracy (%) |
|---|---|---|
| 90 | 39 | 72.9 |
| 95 | 36 | 74.7 |

### (X) Micheck 1a_{Validatedpv} Cross-Validated Model On Whole Patient Population

Model developed to differentiate AgCaP vs NonAgCaP in CaP population
Data for model development: CaP patients only (74 AgCaP, 38 NonAgCaP)
Data for model performance: CaP patients only (110 AgCaP, 195 NOT AgCaP)
Method: cross-validated standard Multivariable Logistic Regression
AUC is 0.84 (0.80-0.89), ROC Curve is shown in FIG. Twenty Six

TABLE 27

| Sensitivity (%) | Specificity (%) | Accuracy (%) |
|---|---|---|
| 90 | 52 | 65.9 |
| 95 | 46 | 63.3 |

### (Y) Assessment Of Micheck 1a_{Validatedpv} Cross-Validated Model On Whole Patient Population

When applied to the whole population using a cutpoint of 95% sensitivity, The MiCheck 1a_{validatedPV} algorithm classifies 210 patients as positive and 103 patients as negative. The breakdown of test results and the NPV for GS≥3+4 and GS≥4+3 are shown below in Table 28. The percentage reduction in biopsies for no CaP, non-AgCaP and AgCaP are shown in FIG. Twenty Seven.

TABLE 28

Performance of V1 MiCheck1a_{validatedPv} on whole patient population
Algorithm Outcomes

| Diagnosis | Of 210 positive MiCheck tests | Of 95 negative MiCheck tests |
|---|---|---|
| No Cancer | 70 | 69 |
| Non Aggressive CaP | 36 | 20 |
| Aggressive CaP | 104 | 6 (5 GS 3+4, 1 GS 4+3 0 GS >4+3) |

46% of unnecessary biopsies saved
NPV (≥3+4) = 93.7
NPV (≥4+3) = 98.9
5.45% GS ≥3+4 cancers delayed diagnosis
1.79 % GS ≥4+3 cancers delayed diagnosis
0% GS ≥8 cancers delayed diagnosis

### (Z) Assessment of MiCheck 1a_{validatedPV} Cross-Validated Model on Patient Population PSA Range 2-10 ng/ml and PSA 4-10 ng/ml

The MiCheck 1a_{validatedPV} model was tested in patients in the PSA range of 2-10 ng/ml and 4-10 ng/ml using the same cutpoint that gives 95% sensitivity in the whole evaluable PSA range population.

The test performance in these groups is shown below in Table 29.

TABLE 29

Performance of MiChecklavalidatedPV on different PSA ranges
Performance of models in different PSA ranges
A. Whole PSA range

| Model | Variables | AUC (95%CI) | Sens (%) | Spec (%) | Acc (%) | % Biopsy saved | PPV |
|---|---|---|---|---|---|---|---|
| PV MiCheek® Prostate 1a val | PV, PSA, %Free PSA, HE4 | 0.84 (0.80-0.89) | 95 | 46 | 63 | 31 | 50 |
| B. PSA range 2-10 ng/ml | | | | | | | |
| PV MiCheck® Prostate 1a val | PV. PSA, %Free PSA. HE4 | 0.81 (0.75-0.87) | 92 | 48 | 61 | 36 | 44 |
| C. PSA range 4-10 ng/ml | | | | | | | |
| PV MiCheck® Prostate 1a val | PV, PSA, %Free PSA, HE4 | 0.80 (0.74-0.86) | 96 | 35 | 56 | 23.3 | 44 |

| A. Whole PSA range | | | | | | |
|---|---|---|---|---|---|---|
| | NPV GS≥3+4 | NPV GS≥4+3 | Delayed GS≥3+4 | Delayed GS≥4+3 | Delayed GS≥8 | Sample Sizes (No CaP/Non Ag Cap/AgCaP) |
| | 93.7 | 98.9 | 5.5 | 1.8 | 0 | 139/56/110 |
| B. PSA range 2–10 ng/ml | | | | | | |
| | 93 | 99 | 7.7 | 2.6 | 0 | 126/52/78 |
| C. PSA range4–10 ng/ml | | | | | | |
| | 93.8 | 100 | 4.1 | 0 | 0 | 94/39/73 |

### (Aa) Development Of Models With Both Dre And Prostate Volume

The effect of including both DRE and prostate volume in logistic regression models was assessed. Prostate volume was collected for 110 AgCaP, 56 Non-AgCaP and 139 NoCaP subjects. Individual analyte AUCs and p values for differentiating non-aggressive cancer or non-aggressive and no cancer patients are shown in Table 19 above.

For each standard Logistic regression model, PSA, %free PSA, PV and HE4 values were obtained and log transformed. The transformed values were multiplied by their respective log odds ratio co-efficient. If an abnormal/suspicious DRE status was obtained, it was multiplied by its log odds ratio co-efficient. The products were summed to generate a Logit(P) value which was then used in the following equation to generate a probability score P.

The General equation is:

$$\begin{matrix}\text{Logit} \left(P\right) = \text{log} \left(P / 1 \text{-} P\right) = \text{intercept} \text{+} \sum \text{log} \text{odds} {\text{ratio}}_{i} \\ \times l o g \left(m a r ker {}_{i}\right) + \sum \text{log} \text{odds} {\text{ratio}}_{D R E} \times 1 \left(\text{if} \text{suspicious} \text{DRE}\right)\end{matrix}$$

$$\begin{matrix}P = \frac{e x p \left(L o g i t \left(P\right)\right)}{1 + e x p \left(L o g i t \left(P\right)\right)} \\ P \text{is} \text{a} \text{value} \text{between} \text{0} \text{and} \text{1} \text{that} \text{indicates} \text{} \text{the} \text{risk} \text{of} \text{AgCaP}\end{matrix}$$

Classification:
   If P > Threshold the patient is classified as having AgCaP

The contribution of additional analytes to the performance of different models is shown in Table 30.

TABLE 30

Comparison of models developed using 1-5 markers in the CaP and Whole evaluable population

| Model | Component(s) | CaP population (166 samples -110 AgCaP vs 56 NonAgCaP) | | CaP model applied to whole population (305 samples - 110 AgCaP vs 195 others) | |
|---|---|---|---|---|---|
| | | AUC (95%Cl) | Specificity at 95% Sensitivity | AUC (95%Cl) | Specificity at 95%Sens |
| (a) | PSA | 0.73 (0.66-0.81) | 27 | 0.73 (0.68 - 0.79) | 31 |
| (b) | DRE | 0.58 (0.52-0.64) | Sens:26, Spec: 89 | 0.58 (0.53 - 0.62) | Sens:26, Spec: 89 |
| (c) | PV | 0.62 (0.53-0.71) | 10 | 0.70 (0.64 - 0.76) | 22 |
| (d) | %free PSA | 0.70 (0.61-0.78) | 14 | 0.76 (0.70 - 0.81) | 27 |
| (e) | HE4 | 0.62 (0.53-0.72) | 13 | 0.59 (0.53 - 0.66) | 16 |
| (f) | PSA, PV | 0.77 (0.70-0.84) | 29 | 0.82 (0.77-0.87) | 44 |
| (g) | PSA, PV, %free PSA | 0.78 (0.71-0.85) | 29 | 0.83 (0.78 - 0.88) | 39 |
| (h) | PSA, PV, %freePSA, HE4 | 0.80 (0.73-0.87) | 36 | 0.85 (0.80 - 0.89) | 45 |
| (k) | PSA, DRE, %freePSA, HE4 | 0.80 (0.73-0.87) | 41 | 0.82 (0.78-0.87) | 48 |
| (i) | PSA, DRE, PV, %freePSA, HE4 | 0.81 (0.75-0.88) | 39 | 0.86 (0.82-0.90) | 49 |

TABLE 31

Comparison of performance of different models in CaP and Whole evaluable population

| Comparison of Models | CaP population | | CaP model applied to whole population | |
|---|---|---|---|---|
| | (166 samples - 110 AgCaP vs 56 NonAgCaP) | | (305 sample -110 AgCaP vs 195 others) | |
| (h v g) (Difference in AUC) | 0.02 | p value = 0.355 | 0.02 | p value = 0.355 |
| (h v g) Difference in specificity at 95%Sens | 7% | p value = 0.289 | 6% | P value = 0.090 |
| (k v h) (Difference in AUC) | 0 | P value = 0.919 | 0.03 | P value = 0.062 |
| (k v h) Difference in specificity at 95%Sens | 5% | P value = 0.505 | 3% | P value = 0.510 |
| (I v h) (Difference in AUC) | 0.01 | P value = 0.258 | 0.01 | P value = 0.184 |
| (I v h) Difference in specificity at 95%Sens | 3% | P value = 0.683 | 4% | P value = 0.230 |
| (I v k) (Difference in AUC) | 0.01 | P value = 0.205 | 0.04 | P value = 0.0005 |
| (I v k) Difference in specificity at 95%Sens | 2% | P value = 0.864 | 1% | P value = 1.00 |

Of note, addition of PV and DRE (model 1) increased the AUC in differentiating AgCaP from non-AgCaP in the CaP population compared to models (h) and (k) (0.81 vs 0.80), while the specificity at 95% sensitivity showed either a small increase (36%-39%) or a small decrease (41% to 39%) for models (h) and (k) respectively. None of these changes reached statistical significance (Table 31).

When model (i) was applied to the whole population, inclusion of both DRE and PV increased the AUC compared to models (h) or (k) (0.86 vs 0.85 and 0.86 vs 0.82 respectively, Table 31) and this was statistically significant for model (i) compared to model (k). Inclusion of both DRE and PV increased the specificity at 95% sensitivity compared to both models (h) and (k) in this population (49% vs 45% and 49% vs 48%) but this did not achieve statistical significance.

### (Bb) Standard Logistic Regression Model 1a Developed on the CaP Population Only

Model developed to differentiate AgCaP vs NonAgCaP in CaP population
Data for model development: CaP patients only (110 AgCaP, 56 NonAgCaP)
Data for performance report: CaP patients only (110 AgCaP, 56 NonAgCaP)
Method: Standard Multivariable Logistic Regression
AUC is 0.81 (0.75-0.88), ROC curve is shown in FIG. Twenty Eight

TABLE 32A

| Variable | Transformation | Log Odd ratio |
|---|---|---|
| (Intercept) | | -5.2904279 |
| Central.PSA | Log | 1.87465288 |
| Prostate Volume | Log | -0.9809664 |
| DRE | | 1.27662837 |
| %Free.PSA | Log | -0.8107134 |
| WFDC2 (HE4) | Log | 0.89546752 |

TABLE 32B

| Sensitivity (%) | Specificity (%) | Accuracy (%) |
|---|---|---|
| 90 | 52 | 77.1 |
| 95 | 39 | 75.9 |

### (Cc) Standard Logistic Regression Model 1a Applied to the Whole Patient Population

The model developed in (bb) was applied to the whole patient population.
Data for model development: CaP patients only (110 AgCaP, 56 NonAgCaP)
Data for performance report: whole evaluable population (110 AgCaP, 195 NOT AgCaP)
Method: Standard Multivariable Logistic Regression
AUC is 0.86 (0.82-0.90), ROC curve is shown in FIG. Twenty Nine

TABLE 33A

| Variable | Transformation | Log Odd ratio |
|---|---|---|
| (Intercept) | | -5.2904279 |
| Central.PSA | Log | 1.87465288 |
| Prostate Volume | Log | -0.9809664 |
| DRE | | 1.27662837 |
| %Free.PSA | Log | -0.8107134 |
| WFDC2 (HE4) | Log | 0.89546752 |

TABLE 33B

| Sensitivity (%) | Specificity (%) | Accuracy (%) |
|---|---|---|
| 90 | 62 | 75.1 |
| 91 | 59 | 70.8 |
| 92 | 57 | 69.8 |
| 93 | 50 | 65.2 |
| 95 | 49 | 65.2 |

### (Dd) Standard Logistic Regression Model 1a Applied To The Psa 2-10 Ng/Ml Cap Patient Population

The model developed in (bb) was applied to the whole patient population.
Data for model development: CaP patients only (110 AgCaP, 56 NonAgCaP)
Data for performance report: CaP population PSA range 2-10 ng/ml (78 AgCaP, 52 NonAgCaP)
Method: Standard Multivariable Logistic Regression
AUC is 0.78 (0.70-0.86), ROC curve is shown in FIG. Thirty

TABLE 34A

| Variable | Transformation | Log Odd ratio |
|---|---|---|
| (Intercept) | | -5.2904279 |
| Central.PSA | Log | 1.87465288 |
| Prostate Volume | Log | -0.9809664 |
| DRE | | 1.27662837 |
| %Free.PSA | Log | -0.8107134 |
| WFDC2 (HE4) | Log | 0.89546752 |

TABLE 34B

| Sensitivity (%) | Specificity (%) | Accuracy (%) |
|---|---|---|
| 90 | 44 | 71.5 |
| 92 | 42 | 72.3 |
| 95 | 38 | 72.3 |

The cutpoint used for 95% sensitivity in the whole population, showed 92% sensitivity in the PSA 2-10 ng/ml population (bolded).

### (Ee) Standard Logistic Regression Model 1a Applied To The Whole Patient Population Psa Range 2-10 Ng/Ml

The model developed in (bb) was applied to the whole patient population.
Data for model development: CaP patients only (110 AgCaP, 56 NonAgCaP)
Data for performance report: whole evaluable population PSA range 2-10 ng/ml (78 AgCaP, 178 NOT AgCaP)
Method: Standard Multivariable Logistic Regression
AUC is 0.84 (0.78-0.89), ROC curve is shown in FIG. Thirty One

TABLE 35A

| Variable | Transformation | Log Odd ratio |
|---|---|---|
| (Intercept) | | -5.2904279 |
| Central.PSA | Log | 1.87465288 |
| Prostate Volume | Log | -0.9809664 |
| DRE | | 1.27662837 |
| %Free.PSA | Log | -0.8107134 |
| WFDC2 (HE4) | Log | 0.89546752 |

TABLE 35B

| Sensitivity (%) | Specificity (%) | Accuracy (%) |
|---|---|---|
| 90 | 52 | 63.7 |
| 92 | 51 | 63.7 |
| 95 | 46 | 60.5 |

The cutpoint used for 95% sensitivity in the whole population, showed 92% sensitivity in the PSA 2-10 ng/ml population (bolded).

### REFERENCES

1. Neal D Shore, Christopher M Pieczonka, R Jonathan Henderson, James L Bailen, Daniel R Saltzstein, Raoul S Concepcion, Jennifer L Beebe-Dimmer, Julie J Ruterbusch, Thao Ho Le, Rachel A Levin, Sandra Wissmueller, Philip Prah, Robert Borotkanics, Thomas A Paivanas, Arletta van Breda, Douglas H Campbell, Bradley J Walsh. A Comparison of Prostate Health Index, Total PSA, %free PSA, and proPSA in a Contemporary US population - The MiCheck-01 Prospective Trial. Urol Oncol. 2020 Apr 29;S1078-1439(20)30098-3. doi: 10.1016/j.urolonc.2020.03.011.Online ahead of print.
2. R Core Team (2017). R: A language and environment for statistical computing. R Foundation for Statistical Computing, Vienna, Austria. ISBN 3-900051-07-0, URL http://www.R-project.org/
3. Adrian Raftery, Jennifer Hoeting, Chris Volinsky, Ian Painter and Ka Yee Yeung (2018). BMA: Bayesian Model Averaging. R package version 3.18.8. https://CRAN.R-project. org/package=BMA
4. Genuer, R. and Poggi, J.M. and Tuleau-Malot, C. (2010), Variable selection using random forests, Pattern Recognition Letters 31(14), 2225-2236
5. Genuer, R. and Poggi, J.M. and Tuleau-Malot, C. (2015), VSURF: An R Package for Variable Selection Using Random Forests, The R Journal 7(2):19-33
6. Max Kuhn. Contributions from Jed Wing, Steve Weston, Andre Williams, Chris Keefer, Allan Engelhardt, Tony Cooper, Zachary Mayer, Brenton Kenkel, the R Core Team, Michael Benesty, Reynald Lescarbeau, Andrew Ziem, Luca Scrucca, Yuan Tang, Can Candan and Tyler Hunt. (2018). caret: Classification and Regression Training. R package version 6.0-79. https://CRAN.R-project.org/package=caret
7. Xavier Robin, Natacha Turck, Alexandre Hainard, et al. (2011) “pROC: an open-source package for R and S+ to analyze and compare ROC curves”. BMC Bioinformatics, 7, 77. DOI: 10.1186/1471-2105-12-77
8. Sing T, Sander O, Beerenwinkel N and Lengauer T (2005). “ROCR: visualizing classifier performance in R.” _Bioinformatics_, *21*(20), pp. 7881. <URL: http://rocr.bioinf.mpi-sb.mpg.de>
9. Doss T, Ahmed K, Raison N, Challacombe B, Dasgupta P. Clarifying the PSA grey zone: the management of patients with a borderline PSA. Int J Clin Practice 2016, 70 (11) p 950-959.

1. A method for diagnosing aggressive prostate cancer (CaP) in a test subject, comprising:
(a) obtaining an analyte level for one or more analytes in the test subject’s biological sample, and obtaining a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject has aggressive CaP by comparison of the subject test score value and the threshold value, wherein:
the one or more analyte/s comprise or consist of WAP four-disulfide core domain protein 2 (WFDC2 (HE4)), and optionally total prostate specific antigen (PSA),
the one or more clinical variables comprise at least one of: %Free PSA, DRE, Prostate Volume (PV) and
the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value.

2. The method of claim 1, wherein the population of control subjects comprises subjects that do not have prostate cancer and subjects that have non-aggressive prostate cancer.

3. A method for discerning whether a test subject has non-aggressive or aggressive prostate cancer (CaP), comprising:
(a) obtaining an analyte level for one or more analytes in the test subject’s biological sample, and obtaining a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject has aggressive CaP by comparison of the subject test score value and the threshold value, wherein:
the one or more analyte/s comprise or consist of WFDC2 (HE4), and optionally total PSA,
the one or more clinical variables comprise at least one of: %Free PSA, DRE, Prostate Volume (PV) and
the threshold value was determined by:
measuring said one or more analyte/s in a series of biological samples obtained from a population of subjects having aggressive CaP and from a population of control subjects having non-aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and non-aggressive CaP, to thereby generate the threshold value.

4. The method of claim 1 or claim 3, wherein the population of control subjects has non-aggressive CaP as defined by a Gleason score of 3+3.

5. The method of any one of claims 1 to 4, wherein the threshold value is determined prior to performing the method.

6. The method of any one of claims 1 to 5, wherein the one or more clinical variables and the one or more analyte/s comprise or consist of any one of the following:
WFDC2 (HE4) and %Free PSA
WFDC2 (HE4) and DRE
WFDC2 (HE4) and PV
WFDC2 (HE4), %Free PSA, and DRE
WFDC2 (HE4), %Free PSA, and PV
WFDC2 (HE4), total PSA and %Free PSA
WFDC2 (HE4), total PSA and DRE
WFDC2 (HE4), total PSA and PV
WFDC2 (HE4), total PSA, %Free PSA, and DRE, or
WFDC2 (HE4), total PSA, %Free PSA, and PV.

7. The method of any one of claims 1 to 6, comprising selecting a subset of the combined analyte/s and/or clinical variable measurements to generate the threshold value.

8. The method of any one of claims 1 to 7, wherein said combining of each said analyte level of the series with said measurements of the one or more clinical variables comprises combining a logistic regression score of the clinical variable measurements and analyte level/s in a manner that maximizes said discrimination, in accordance with the formula:
$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times t r a n s f o r m e d \left(v a r i a b l {e}_{i}\right)\right) \\ P = \frac{e x p \left(L o g i t \left(P\right)\right)}{1 + e x p \left(L o g i t \left(P\right)\right)}\end{matrix} \qquad \text{(i)}$$
wherein:
P is probability of that the test subject has aggressive prostate cancer,
the coefficientᵢ is the natural log of the odds ratio of the variable,
the transformed variableᵢ is the natural log of the variableᵢ value; or
$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times \right) \\ t r a n s f o r m e d \left(v a r i a b l {e}_{i}\right) + c o e f f i c i e n {t}_{D R E} \times D R E \\ P = \frac{e x p \left(L o g i t \left(P\right)\right)}{1 + e x p \left(L o g i t \left(P\right)\right)}\end{matrix} \qquad \text{(ii)}$$
wherein:
P is probability that the test subject has aggressive prostate cancer,
the coefficientᵢ is the natural log of the odds ratio of the variable,
the transformed variableᵢ is the natural log of the variableᵢ value,
a DRE value of 1 indicates abnormal, while DRE value of 0 indicates normal.

9. The method of any one of claims 1 to 8, wherein said applying a suitable algorithm and/or transformation to the combination of the clinical variable measurements and analyte level/s comprises use of an exponential function, a logarithmic function, a power function and/or a root function.

10. The method according to any one of claims 1 to 9, wherein the suitable algorithm and/or transformation applied to the combination of the clinical variable measurements and analyte level/s of the test subject is in accordance with the formula:
$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times t r a n s f o r m e d \left(v a r i a b l {e}_{i}\right)\right) \\ P = \frac{e x p \left(L o g i t \left(P\right)\right)}{1 + e x p \left(L o g i t \left(P\right)\right)}\end{matrix} \qquad \text{(i)}$$
wherein:
P is probability of that the test subject has aggressive prostate cancer,
the coefficientᵢ is the natural log of the odds ratio of the variable,
the transformed variableᵢ is the natural log of the variableᵢ value; or
$$\begin{matrix}\text{Logit} \left(\text{P}\right) = \text{Log} \left(\text{P} / 1 \text{-P}\right) \\ = i n t e r c e p t + {\sum }_{i = 1}^{\text{N}} \left(c o e f f i c i e n {t}_{i} \times \right) \\ t r a n s f o r m e d \left(v a r i a b l {e}_{i}\right) + c o e f f i c i e n {t}_{D R E} \times D R E \\ P = \frac{e x p \left(L o g i t \left(P\right)\right)}{1 + e x p \left(L o g i t \left(P\right)\right)}\end{matrix} \qquad \text{(ii)}$$
wherein:
P is probability of that the test subject has aggressive prostate cancer,
the coefficientᵢ is the natural log of the odds ratio of the variable,
the transformed variableᵢ is the natural log of the variableᵢ value,
a DRE value of 1 indicates abnormal, while DRE value of 0 indicates normal;
and wherein said suitable algorithm and/or transformation is used to generate the subject test score that is compared to the threshold value to thereby determine whether or not the test subject has aggressive prostate cancer.

11. The method according to any one of claims 1 to 10, wherein said combining of each said analyte level of the series with measurements of said one or more clinical variables obtained from each said subject of the populations maximizes said discrimination.

12. The method of any one of claims 1 to 11, wherein said combining of each said analyte level of the series with the measurements of one or more clinical variables obtained from each said subject of the populations is conducted in a manner that:
(i) reduces the misclassification rate between the subjects having aggressive CaP and said control subjects; and/or
(ii) increases sensitivity in discriminating between the subjects having aggressive CaP and said control subjects; and/or
(iii) increases specificity in discriminating between the subjects having aggressive CaP and said control subjects.

13. The method of claim 12, wherein said combining in a manner that reduces the misclassification rate between the subjects having aggressive CaP and said control subjects comprises selecting a suitable true positive and/or true negative rate.

14. The method of claim 12, wherein said combining in a manner that reduces the misclassification rate between the subjects having aggressive CaP and said control subjects minimizes the misclassification rate.

15. The method of claim 12, wherein said combining in a manner that reduces the misclassification rate between the subjects having aggressive CaP and said control subjects comprises minimizing the misclassification rate between the subjects having aggressive CaP and said control subjects by identifying a point where the true positive rate intersects the true negative rate.

16. The method of claim 12, wherein said selecting the threshold value from the combined clinical variable measurement/s and combined analyte level/s in a manner that increases sensitivity in discriminating between the subjects having aggressive CaP and said control subjects increases or maximizes said sensitivity.

17. The method of claim 12, wherein said selecting the threshold value from the combined clinical variable measurement/s and combined analyte level/s in a manner that increases specificity in discriminating between the subjects having aggressive CaP and said control subjects increases or maximizes said specificity.

18. The method according to any one of claims 1 to 17, wherein the one or more clinical variables and the one or more analytes comprise or consist of:
total PSA, %free PSA, DRE, WFDC2 (HE4)
total PSA, %free PSA, PV, WFDC2 (HE4), or
total PSA, %free PSA, DRE, PV, WFDC2 (HE4).

19. The method according to any one of claims 1 to 18, wherein the test subject has previously received a positive indication of prostate cancer.

20. The method according to any one of claims 1 to 19, wherein the test subject has previously received a positive indication of prostate cancer by digital rectal exam (DRE) and/or by PSA testing.

21. The method according to any one of claims 1 to 19, wherein the test subject has a PSA level of 2-10 ng/mL blood, or 4-10 ng/mL blood.

22. The method according to any one of claims 1 to 21, wherein the series of biological samples obtained from each said population and/or the test subject’s biological sample are selected from; whole blood, serum, plasma, saliva, tear/s, urine, and tissue.

23. The method according to any one of claims 1 to 22, wherein said test subject, said population of subjects having aggressive CaP, and said population of control subjects are human.

24. The method of any one of claims 1 to 23, further comprising measuring one or more analyte/s in the test subject’s biological sample to thereby obtain the analyte level for each said one or more analytes.

25. The method according to claim 24, wherein said measuring of one or more analyte/s in the test subject’s biological sample comprises:
(i) measuring one or more fluorescent signals indicative of each said analyte level;
(ii) obtaining a measurement of weight/volume of said analyte/s in the biological sample;
(iii) measuring an absorbance signal indicative of each said analyte level; or
(iv) using a technique selected from the group consisting of: electrochemiluminescence, mass spectrometry, a protein array technique, high performance liquid chromatography (HPLC), gel electrophoresis, radiolabeling, and any combination thereof.

26. The method according to claim 24 or claim 25, wherein the test subject’s biological sample is contacted, or the series of biological samples was contacted, with first and second antibody populations for detection of each said analyte, wherein each said antibody population has binding specificity for one of said analytes, and the first and second antibody populations have different analyte binding specificities.

27. The method according to claim 26, wherein the first and/or second antibody populations are labelled.

28. The method according to claim 27, wherein the first and/or second antibody populations comprise a label selected from the group consisting of a radiolabel, a fluorescent label, a biotin-avidin amplification system, a chemiluminescence system, microspheres, and colloidal gold.

29. The method according to any one of claims 26 to 28, wherein binding of each said antibody population to the analyte is detected by a technique selected from the group consisting of: immunofluorescence, radiolabeling, immunoblotting, Western blotting, enzyme-linked immunosorbent assay (ELISA), flow cytometry, immunoprecipitation, immunohistochemistry, biofilm test, affinity ring test, antibody array optical density test, and chemiluminescence.

30. The method of any one of claims 24 to 29, wherein said measuring of each said analyte in the biological sample from the test subject or the series of biological samples obtained from each said population comprises measuring the analytes directly.

31. The method of any one of claims 24 to 29, wherein said measuring of each said analyte in the biological sample from the test subject or the series of biological samples obtained from each said population comprises detecting a nucleic acid encoding the analytes.

32. The method of any one of claims 1 to 31, further comprising measuring the two one or more clinical variables in the test subject.

33. The method of any one of claims 1 to 32, further comprising determining said threshold value.

34. The method of claim 33, wherein determining said threshold value comprises measuring said one or more analyte/s in a series of biological samples obtained from a population of subjects having aggressive CaP and from a population of control subjects not having aggressive CaP, to thereby obtain an analyte level for each said analyte in each said biological sample of the series.
